(19) United States
(12) Patent Application Publication
(10) Pub. No.: US 2011/0288063 A1
(43) Pub. Date: Nov. 24, 2011

(54) NOVEL FUSED BRIDGED BICYCLIC HETEROARYL SUBSTITUTED 6-ALKYLIDENE PENEMS AS POTENT BETA-LACTAMASE INHIBITORS

(75) Inventors: Samarendra N. MAITI, Edmonton (CA); Rong Ling, Edmonton (CA); Judy Yip, Edmonton (CA); Chuanjun Gao, Edmonton (CA); Dai Nguyen, Edmonton (CA); Biswajeet Ganguli, Edmonton (CA); Hong Liang, Edmonton (CA); Jehangir Khan, Edmonton (CA); Andhe V. Narender Reddy, Edmonton (CA)

(73) Assignee: Naeja Pharmaceutical Inc., Edmonton (CA)

(21) Appl. No.: 13/111,156

(22) Filed: May 19, 2011

Related U.S. Application Data
(60) Provisional application No. 61/346,184, filed on May 19, 2010.

(51) Int. Cl. A61K 31/431; C07D 499/861; A61P 31/04; C07D 499/08

(52) U.S. Cl. 514/193; 540/304

(57) ABSTRACT

A compound of formula (I) or formula (Ia)

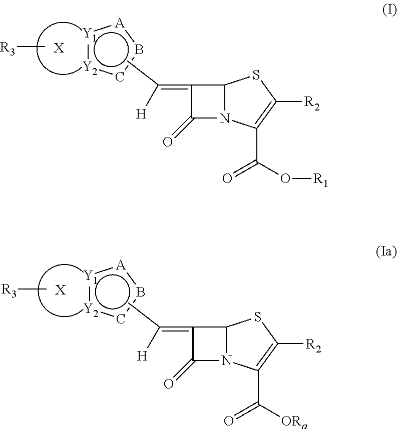

Wherein R₁, Rₐ, R₂, X, R₃, Y₁, Y₂, A, B and C are as defined herein. Also, pharmaceutical compositions comprising such compounds and excipients, methods of treating bacterial infections comprising administering such compounds, methods for making such compounds and hydrates of such compounds.

### CROSS-REFERENCE TO RELATED APPLICATIONS

This application claims the benefit of U.S. Provisional Patent Application No. 61/346,184, filed May 19, 2010, the entirety of which is incorporated herein by reference as if set forth in its entirety.

### FIELD OF INVENTION

This invention relates to novel fused bridged bicyclic heteroaryl substituted 6-alkylidene penem derivatives, which are of value as broad-spectrum β-lactamase inhibitors for use in combination with β-lactam antibiotics to increase their effectiveness in infections caused by β-lactamase producing bacteria.

### BACKGROUND OF THE INVENTION

Microbial drug resistance is an unavoidable consequence resulting from abuse and overuse of antimicrobial agents. The rate at which resistance arises among microbial population is often dictated by the extent of use of particular agents in a given environment. Given the degree of popularity of β-lactam antibiotics it is not surprising that the prevalence of β-lactamase producing strains is increasing worldwide. The most significant known mechanism related to the development of bacterial resistance to the β-lactam antibiotics is the production of class A, class B, class C and class D β-lactamases. Class-A enzymes preferentially hydrolyze penicillins, class-B enzymes hydrolyze preferentially carbapenems, class-C enzymes have a substrate profile favoring cephalosporin hydrolysis; whereas class-D β-lactamases hydrolyze preferentially oxacillin. The commercially available β-lactamase inhibitors such as clavulanic acid, sulbactam and tazobactam are all effective against class-A producing pathogens, but ineffective against class C producing organisms, newly emerged extended-spectrum β-lactamases (ESBLs) and carbapenem resistant KPC type enzymes. Thus, there is a need for a new generation of broad spectrum β-lactamase inhibitors.

EP 0 041 768, EP 0 120613, EP 0 150 781, EP 0 154 132, EP 0 210 065, US 1984/448 5110, WO 87/00525, WO 94/10178, EP 02 10814, EP 02 32966, EP 291304, EP 321186, EP 321187, WO 93/03042, WO 94/22819, WO 95/17184, WO 95/28935, WO 03/93279, WO 03/93280, US 2004/0214812, WO 06/130588, WO 07/30166, WO 07/16134, WO 07/27323, WO 03/93277 and US 2004/0132708 disclose 6-alkylidene penems as β-lactams inhibitors.

### DETAILED. DESCRIPTION OF THE INVENTION

The present invention is directed to fused bridged bicyclic heteroaryl substituted 6-alkylidene penems, which have β-lactamase inhibitory activity. These compounds when used in combination with a β-lactam antibiotic prevent the hydrolysis of the β-lactam agents and thereby enhance the antibacterial properties. These compounds are particularly suitable in combination with a β-lactam antibiotic for the treatment of diseases, conditions or disorders caused by β-lactamase producing organisms. The compounds of the present invention are therefore useful in the treatment of antibacterial infections in humans or animals in combination with other antibiotics. The present invention relates to a compound of formula (I) and (Ia).

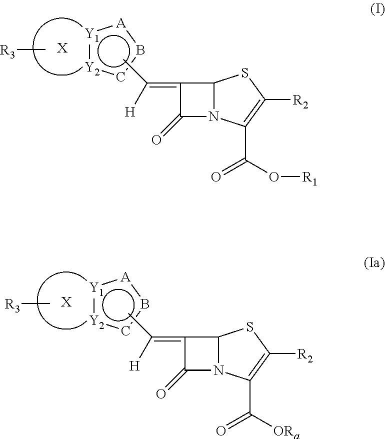

It is an objective of the current invention to provide novel and new bridged bicyclic heteroaryl substituted 6-alkylidene penems (I) having β-lactamase inhibitory action.

It is another object of the invention to provide processes for preparing the same. It is a further object of the invention to provide pharmaceutical compositions comprising a β-lactamase inhibitor (I) or (Ia) of this invention in combination with a β-lactam antibiotic and a pharmaceutically acceptable carrier or diluent.

It is an additional object of the invention to provide an improved method for the treatment of bacterial infections caused by β-lactamase producing bacteria in mammalian subjects.

R₁ in the formula (I) is a carboxy protecting group acceptable in the synthesis of β-lactam chemistry. A variety of protecting groups conventionally used in the β-lactam field to protect carboxy groups can be used for R₁. The major requirements for the protecting group is that it can be removed without cleaving the β-lactam ring and is sufficiently stable under the reaction conditions to permit easy access to the compound of formula (Ia). Examples of most commonly used ester protecting groups are: 4-nitrobenzyl, 4-methoxybenzyl, allyl, 2,2,2-trichloroethyl etc.

This invention also includes a pharmaceutically acceptable ester residue of the compounds of the formula (Ia), which is easily hydrolyzed in vivo. These types of esters are now quite conventional for β-lactam compounds. In general, these non-poisonous esters are cleaved rapidly under physiological conditions producing the non-toxic pharmaceutically acceptable free acid of the formula (Ia) in which Rₐ is hydrogen atom. In many instances, formation of such an ester from a carboxylic acid improves the oral absorption of the parent acid. U.S. Pat. No. 4,446,144 and EP 13,617 describes a number of ester-forming radicals, which give pharmaceutically acceptable esters readily hydrolyzable in vivo. Examples of such groups for forming a readily in vivo hydrolyzable ester groups represented by Rₐ in the formula (Ia) include: pivaloyloxymethyl, acetoxymethyl, α-acetoxyethyl, α-acetoxybenzyl, α-pivaloyloxyethyl, ethoxycarbonyloxymethyl, dimethylaminomethyl, diethylaminoethyl, dimethylaminoethyl, phthalidyl, dimethoxyphthalidyl, (1-ethoxycarbonyloxy)ethyl, 4-crotonolactonyl, gamma-butyrolacton-4-yl, and the like. Preferred individual esters readily hydrolyzable in vivo of the β-lactamase inhibitors of formula (Ia) are selected from the following fragments:

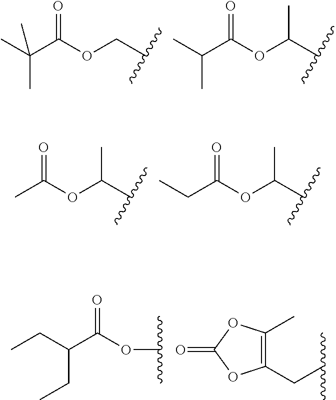

Examples of the groups for forming a pharmaceutically acceptable salt represented by Rₐ in the formula (Ia) include inorganic base salts, ammonium salts, organic base salts, basic amino acid salts, inorganic acid addition salts, and organic acid addition salts. Inorganic bases that can form the inorganic base salts include alkali metals (e.g. sodium, potassium, and lithium) and alkaline earth metals (e.g. calcium and magnesium). Organic bases that can form the organic base salts include n-propylamine, n-butylamine, cyclohexylamine, benzylamine, octylamine, ethanolamine, diethanolamine, diethylamine, triethylamine, dicyclohexylamine, procaine, choline, N-methylglucamine, morpholine, pyrrolidine, piperidine, N-ethylpiperidine and N-methylmorpholine. Basic amino acids that can form the basic amino acid salts include lysine, arginine, ornithine and histidine. As will be appreciated by one skilled in the art, the compounds of formula (Ia) containing a basic nitrogen atom are capable of forming acid addition salts. Such salts with pharmaceutically acceptable acids are included in the invention. Examples of such acids are hydrochloric, hydrobromic, phosphoric, sulfuric, citric, oxalic, maleic, fumaric, tartaric, succinic, malic, formic, acetic, trifluoroacetic, methanesulfonic, trifluoromethanesulfonic, benzenesulfonic, p-toluenesulfonic, 2-naphthalenesulfonic, and the like.

Moreover, when Rₐ is hydrogen in the formula (Ia), it can form a zwitterion by interacting with a basic nitrogen atom present in the molecule.

R₂ in the formulas (I) and (Ia) represents:

(a) hydrogen
(b) straight or branched chain alkyl
(c) hydroxymethyl
(d) alkoxymethyl
(e) aminocarbonyloxymethyl
(f) aryl
(g) heteroaryl
(h) heterocyclyl

Heteroaryl means a 5-(or 6-) membered unsaturated aromatic ring containing from 1 to 4 of any one or more of the hetero atoms selected from O, S and N. Preferred heteroaryl rings include thienyl, furyl, pyridyl, pyrimidinyl and the like.

Heterocyclyl means a 5-membered saturated ring containing one heteroatom, preferably O. The preferred heterocyclyl ring is tetrahydrofuryl.

In the formulas (I) and (Ia), X is a bridged bicyclic ring system having optionally one or two heteroatoms selected from O, N and S. The preferred heteroatoms are O and N.

The said ring X in the formulas (I) and (Ia) may be optionally substituted with R₃, wherein R₃ is selected from:

(a) hydrogen
(b) alkyl
(c) hydroxyl
(d) alkoxy
(e) hydroxymethyl
(f) alkoxymethyl
(g) halogen
(h) cyano
(i) carboxy
(j) alkoxycarbonyl
(k) amino
(l) aminoalkyl
(m) mono- or dialkylamino
(n) mono- or dialkylamino alkyl
(o) acylamino
(p) sulfonylamino
(q) substituted or unsubstituted amidino
(r) substituted or unsubstituted urea
(s) substituted or unsubstituted thiourea
(t) substituted or unsubstituted carboxamido
(u) substituted or unsubstituted thiocarboxamido
(v) substituted or unsubstituted aryl
(w) substituted or unsubstituted aralkyl
(x) substituted or unsubstituted heteroaryl
(y) substituted or unsubstituted heteroarylalkyl
(z) substituted or unsubstituted heterocyclylalkyl.

The heteroaryl group mentioned in items (x) and (y) means 5-(or 6-) membered unsaturated aromatic ring containing from 1 to 4 of any one or more of the heteroatoms selected from O, S and N, where the said heteroaryl groups could be bonded via carbon or a nitrogen containing heteroaryl group could be bonded via nitrogen.

Examples of the heterocyclic rings under item (z) are:

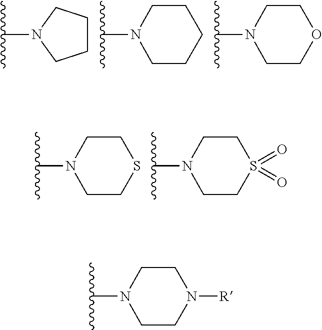

Furthermore, the bridged bicyclic ring systems containing a NH ring atom may be optionally substituted on the said nitrogen by a substituent selected from:

(a) alkyl
(b) alkenyl
(c) alkynyl
(d) cycloalkyl
(e) cycloalkylalkyl
(f) cycloalkenyl
(g) cycloalkenylalkyl
(h) aryl
(i) arylalkyl
(j) heteroaryl
(k) heteroarylalkyl
(l) heterocyclyl
(m) heterocyclylalkyl
(n) or a protecting group like the tert-butylcarbonyloxy.

Y₁ and Y₂ may independently be C or N

In the formulas (I) and (Ia), A, B or C form part of a heteroaryl ring where one of A, B or C is a carbon atom to which the remainder of the molecule is attached.

A, B and C are independently selected from CR₄, O, N, S or NR₅.

In another aspect where A, B and C form part of the heteroaryl ring, the said heretoaryl ring is imidazole, pyrazole, thiazole, oxazole, pyrrole, furan or thiophene ring. In particular the heteroaryl ring is pyrazole and imidazole.

R₄ is hydrogen or lower alkyl.

More preferably, R₄ is hydrogen.

Preferred groups for R₅ are:

(a) hydrogen
(b) straight or branched lower alkyl
(c) lower alkenyl
(d) lower alkynyl
(e) hydroxy alkyl
(f) alkoxy alkyl
(g) aminocarbonyloxy alkyl
(h) cyano alkyl
(i) amino alkyl
(j) mono- or dialkylaminoalkyl
(k) alkoxycarbonylalkyl
(l) carboxy alkyl
(m) substituted or unsubstituted carboxamidoalkyl
(n) cycloalkyl alkyl
(o) substituted or unsubstituted thiocarboxamidoalkyl
(p) substituted or unsubstituted amidinoalkyl
(q) substituted or unsubstituted guanidinoalkyl
(r) substituted or unsubstituted aminocarbonylaminoalkyl
(s) acylaminoalkyl
(t) aralkyl
(u) heteroarylalkyl
(v) heterocyclyl alkyl

Even more preferably, R₅ group represents:

Methyl, ethyl, propyl, butyl, t-butyl, allyl, propargyl, isopropyl, cyclopropylmethyl, cyclopentylmethyl, hydroxyethyl, methoxyethyl, cyanomethyl, aminoethyl, amidinomethyl, guanidinoethyl, amidinoethyl, CH₂COOC₂H₅, CH₂COOH, carboxamidomethyl, substituted carboxamidomethyl, benzyl, thienylmethyl, furylmethyl, imidazolylmethyl, pyridinylmethyl, 2-piperidinylmethyl, (N-methylpyrrolidinyl)ethyl and the like.

Examples of β-lactam antibiotics which can be used in combination with the compounds of the present invention represented by formulas (I) and (Ia) are commonly used penicillins, such as amoxicillin, ampicillin, azlocillin, mezlocillin, apalcillin, hetacillin, bacampicillin, carbenicillin, sulbenicillin, ticarcillin, piperacillin, mecillinam, methicillin, ciclacillin, talampicillin, oxacillin, cloxacillin, dicloxacillin, and commonly used cephalosporins, such as cephalothin, cephaloridine, cefaclor, cefadroxil, cefamandole, cefazolin, cephalexin, cephradine, cephapirin, cefuroxime, cefoxitin, cephacetrile, cefotiam, cefotaxime, cefatriazine, cefsulodin, cefoperazone, ceftizoxime, cefmenoxime, cefmetazole, cephaloglycin, cefonicid, cefodizime, cefpirome, cefepime, ceftazidime, cefpiramide, ceftriaxone, cefbuperazone, cefprozil, cefixime, ceftobiprole, ceftaroline, cefalonium, cefminox, ceforanide, cefuzonam, cefoxitin, cefotetan, loracarbef, cefdinir, cefditoren, cefetamet, cefcapene, cefdaloxime, ceftibuten, cefroxadine and the like.

The compounds of the present invention represented by formulas (I) and (Ia) include pharmaceutically acceptable solvate thereof, a regioisomer thereof, a stereoisomer thereof or a N-oxide thereof.

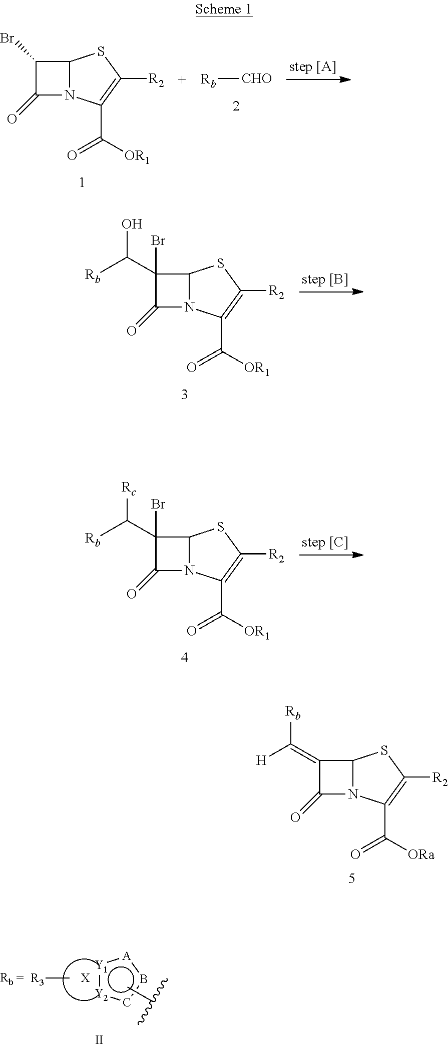

The penem derivatives of the present invention having the formula (I) and (Ia) can be prepared by the process shown in the Scheme 1. The processes differ according to the kind of aldehydes R_{b}CHO used to prepare the penem derivatives.

The fragment R_{b} in the substituted aldehyde 2 is represented by the general structural formula (II), wherein X, Y₁, Y₂, A, B, C and R₃ are as defined before. The steps (A), (B) and (C) of the foregoing process will be described below in details.

### Step (A)

Compounds of the general formula (I) can be prepared by condensing an appropriately substituted aldehyde R_{b}CHO 2 with 6-bromopenem (R₂═H) derivative of structure 1 in presence of a Lewis acid, preferably anhydrous magnesium halide, even more preferably anhydrous magnesium bromide or magnesium bromide etherate and a base in an aprotic medium. The base employed will be a base of low nucleophilicity so that in general primary and secondary amines will not be suitable. Suitable bases include triethylamine, dimethylaminopyridine or diisopropylethylamine. The organic solvents useful in the reaction are not particularly limited and include any of those which do not adversely affect the aldol condensation. Typical solvents are ether, tetrahydrofuran, acetonitrile and the like. The reaction is normally carried out at a temperature of from about −20° C. to about 30° C. and preferably from about −20° C. to −40° C. After completion of the reaction the desired product can be easily separated by conventional methods such as column chromatography, crystallization or similar methods.

### Step (B)

The intermediate aldol product 3 can be functionalized with acid chlorides or acid anhydrides, preferably to an acetate, mesylate or a triflate 4 (R_{c}═OCOCH₃, OSO₂CH₃, OSO₂CF₃). The reagents useful for carrying out this step are acetic anhydride, acetyl chloride, p-toluene sulfonyl chloride, methane sulfonyl chloride, trifluoromethane sulfonyl chloride and the like; preferably acetic anhydride. The reaction is usually conducted in a conventional inert aprotic organic solvent such as tetrahydrofuran, dioxane, acetonitrile, chloroform, dichloromethane, ethyl acetate, dimethyl sulfoxide, pyridine or any other solvent, which does not adversely influence the reaction or a mixture thereof and the reaction time is not critical. The reaction may be carried out at a low temperature such as 0° C. and then to allow the temperature to rise to about to room temperature.

### Step (C)

Compound 4 could be smoothly converted to the desired acid 5 by a reductive elimination process using activated zinc and phosphate buffer at 20° C. to 40° C. at a pH of 6.0 to 8.0. When the protecting group is p-nitrobenzyl substituent then the reductive elimination and deprotection could be achieved by a single step. The elimination and the hydrolysis of the ester protecting group must of course be carried out under conditions to which the groups on the rest of the molecule are stable. Alternatively, compound 4 could be converted to compound 5 under an atmosphere of hydrogen or hydrogen mixed with an inert diluent such as nitrogen or argon in the presence of a hydrogenation catalyst. The catalysts used in this hydrogenation reaction are the type of agents known in the art for this kind of transformation and typical examples are the noble metals, such as nickel, palladium, platinum and rhodium. Examples of the catalysts are platinum, platinum oxide, palladium, palladium oxide, nickel oxide, Raney nickel and the like. The catalyst is usually present in the amount from about 1 to about 50 weight percent and preferably from about 5 to about 10 weight percent based on the compound of formula (I). It is often convenient to suspend the catalyst on an inert support. A particularly convenient catalyst is palladium suspended on an inert support such as carbon, e.g. 10% by weight palladium on carbon. This reaction may be conveniently effected at ambient temperature at 40 psi. Suitable solvents for this reaction are those which substantially dissolve the starting material of the formula (I), are sufficiently volatile to be removed by evaporation and do not themselves suffer hydrogenation. Examples of such solvents include ethanol, dioxane, tetrahydrofuran or a mixture of these solvents and 6.5 phosphate buffer. The penem derivatives of the present invention having the formula (Ia) in which Rₐ is hydrogen can be purified by standard procedures known in the art, such as recrystallization or chromatography e.g. chromatography on HP-20 column. Advantageously, during the reductive elimination step in the present invention the desired “Z” isomer is formed preferentially. The intermediate 1, where R₂ is hydrogen and R₁ is p-nitrobenzyl, may be prepared from the commercially available 6-aminopenicillanic acid (6-APA) by following the patent literature WO 03/093277.

The β-lactamase inhibitors of this invention of formula (Ia) are acidic and they will form salts with basic agents. It is necessary to use a pharmaceutically acceptable non-toxic salt. Moreover, when Rₐ is hydrogen, a compound of formula (Ia) is a mono-acid and will form a mono-salt. Alternatively, it can form an internal salt (zwitterion) by interaction with a basic nitrogen atom present in the molecule of formula (Ia). However, when Rₐ is hydrogen and R₃ contains a carboxylic acid group, the compound of the formula (Ia) is a diacid and can form disalts. In the latter case, the two cationic counter ions can be the same or different. Salts of the compounds of formula (Ia) can be prepared by standard methods known in the penicillin and cephalosporin literature. Typically, this involves contacting the acidic and basic components in the appropriate stoichiometric ratio in an inert solvent system, which can be aqueous, non-aqueous or partially aqueous as appropriate. Favorable pharmaceutically acceptable salts of the compounds of formula (Ia) are sodium, potassium and calcium salts.

In regard to esters readily hydrolyzable in vivo of a compound of the formula (Ia) it is necessary to use a pharmaceutically-acceptable non-toxic ester. Such esters are prepared by standard methods with the specific method being chosen being dependent upon the precise ester to be prepared. If the ester residue is, for example, 3-phthalidyl, 4-crotonolactonyl, gamma-butyrolacton-4-yl, or the like, the esters can be prepared by alkylation of a carboxylate salt of a compound of formula (Ia) with 3-halogenated phthalide, 4-halogenated crotonolactone, 4-halogenated gamma-butyrolactone or the like. The reaction is carried out by dissolving the salt of the penem derivative of the formula (Ia) in a suitable polar organic solvent, such as N,N-dimethylformamide, and then adding about 1 molar equivalent of the halide. Suitable salts of the penem derivative to be used in the esterification are salts of sodium, potassium or like alkali metals; salts of triethylamine, diisopropylethylamine, N,N-dimethylaniline, N-methyl morpholine or like tertiary amines. The reaction temperature ranges from about 0° C. to about 100° C., preferably from about 15° C. to about 25° C. After completion of the reaction, the desired product can be easily separated by conventional methods and purified, when required by recrystallization, column chromatography or the like.

The compounds of the present invention including the pharmaceutically-acceptable salts thereof, and the pharmaceutically-acceptable readily in vivo hydrolyzable esters thereof are inhibitors of bacterial β-lactamases. The compounds increase the antibacterial effectiveness of β-lactamase susceptible β-lactam antibiotics—that is, they increase the effectiveness of the antibiotic against infections caused by β-lactamase producing microorganisms in mammalian subjects. This makes the compounds of formula (Ia) and said pharmaceutically-acceptable salts and esters thereof, valuable for co-administration with β-lactam antibiotics in the treatment of bacterial infections in mammalian subjects, particularly humans. In the treatment of a bacterial infection, said compounds of formula (Ia) or salt or ester thereof can be mixed with the β-lactam antibiotic, and the two agents thereby administered simultaneously. Alternatively, the compound of formula (Ia) or salt or ester thereof can be administered as a separate agent during a course of treatment with the antibiotic.

A compound of formula (Ia) or salt or ester thereof can be administered orally or parenterally. The salts of the compounds of formula (Ia) tend to be more effective when administered parenterally. Whereas in many instances formation of an ester, readily hydrolyzable in vivo increases oral effectiveness. The compounds of the present invention can be administered alone or may be mixed with a pharmaceutically-acceptable carrier or diluent depending on the mode of administration. For oral mode of administration a compound of this invention can be used in the form of tablets, capsules, granules, powders, lozenges, troches, syrups, elixirs, solution, suspensions and the like, in accordance with the standard pharmaceutical practice.

The parenteral administration, which includes intramuscular, intraperitonial, subcutaneous and intravenous use, sterile solutions of the active ingradient are usually prepared, and the pH of the solutions are suitably adjusted and buffered. For intravenous use; the total concentration of solutes should be controlled to render the preparation isotonic.

Carriers useful in formulating the preparations are commonly used pharmaceutically-acceptable non-toxic carriers such as gelatin, lactose, sodium citrate, salts of phosphoric acid, starch, magnesium stearate, sodium lauryl sulfate, talc, polyethylene glycol etc. The carrier may be used with other additives such as diluents, binders, buffer agents, preservatives, sweetening agents, flavoring agents, glazes, disintegrators, coating agents, emulsifying agents, suspending agents etc. In a pharmaceutical composition containing a compound of this invention, the weight ratio of active ingredient to carrier will normally be in the range of 1:20 to 20:1.

The daily dose of the preparation can be appropriately determined and is not particularly limited. However in most instances, an effective β-lactamase inhibiting dose of a compound of formula (Ia) or pharmaceutically acceptable salt or ester thereof, will be a daily dose in the range from about 1 to about 500 mg per kilogram of body weight orally, and from about 1 to about 500 mg per kilogram of body weight parenterally. The weight ratio of the compound of present invention and the β-lactam antibiotic with which it is being administered will normally be in the range from 1:20 to 20:1.

The following examples are provided to demonstrate the operability of the present invention. The structures of the compounds were established by the modes of synthesis and by extensive high field nuclear magnetic resonance spectral techniques and mass spectrophotometry.

### Example 1

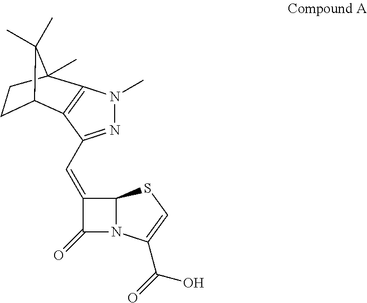

### Preparation of 2-hydroxy(4,7,7-trimethyl-3-oxobicyclo[2.2.1]hept-2-ylidene) ethanoic acid (3)

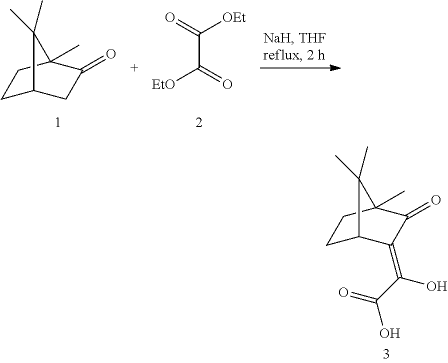

To a solution of (±)-camphor 1 (10.0 g, 65.6 mmol) and diethyl oxalate 2 (17.8 mL, 131.3 mmol) in THF (150 mL) was added NaH (60%, 5.77 g, 144.3 mmol) at room temperature in portions. The resulting mixture was refluxed for 2 h. After cooling to room temperature, the reaction mixture was quenched with crushed ice (200 mL), extracted with Et₂O (1×100 mT) and the layers were separated. The aqueous layer was acidified with 6N HCl aqueous solution to pH 2.0, and extracted with EtOAc (3×100 mL). The combined EtOAc layers were washed with brine, dried over Na₂SO₄ and concentrated under reduced pressure. The residue was recrystallized from hexane to provide 3 (8.0 g, 54%) as a white solid.

¹H NMR (400 MHz, CDCl₃): δ 0.89 (3H, s), 1.03 (3H, s), 1.09 (3H, s), 1.48-1.57 (2H, m), 1.89 (1H, m), 2.13 (1H, m), 3.11 (1H, d, J=3.9 Hz).

### Preparation of ethyl 1,7,8,8-tetramethyl-4,5,6,7-tetrahydro-1H-4,7methanoindazole-3-carboxylate (5)

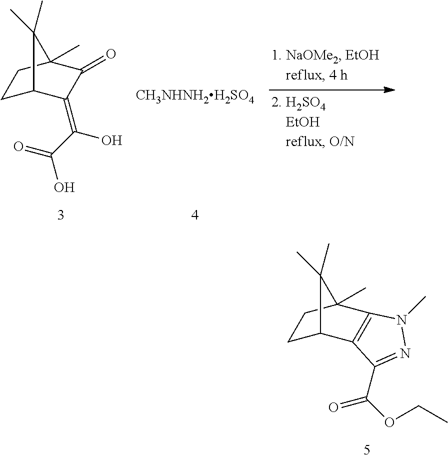

To a suspension of methylhydrazine sulfate 4 (2.56 g, 17.8 mmol) in EtOH (50 mL) was added 25-30% NaOMe/MeOH (6.6 mL, 35.6 mmol, as 30% w/w) slowly. The resulting mixture was stirred at room temperature for 5 min, and then 2-hydroxy(4,7,7-trimethyl-3-oxobicyclo[2.2.1]hept-2-ylidene) ethanoic acid 3 (2.0 g, 8.9 mmol) was added. The resulting mixture was heated under reflux for 4 h and cooled to room temperature, H₂SO₄ (3.0 mL, 55.7 mmol) was added slowly. The reaction mixture was then heated under reflux overnight and concentrated under reduced pressure. The residue was dissolved in EtOAc (50 mL), poured into ice water (100 mL), adjusted pH to 8 with saturated NaHCO₃ aqueous solution and separated. The aqueous layer was extracted with EtOAc (2×20 mL). The combined EtOAc layers were washed with brine, dried over Na₂SO₄ and concentrated under reduced pressure. The residue was purified by column chromatography (silica gel, hexane:EtOAc, 3:1 to 1:1) to afford 5 (1.6 g, 69%) as yellow oil which contained small amount (<5%) of methyl ester.

¹H NMR (400 MHz, CDCl₃): δ 0.76 (3H , s), 0.92 (3H, s), 1.07-1.19 (2H, m), 1.35 (3H, s), 1.38 (3H, t, J=0.6 Hz), 1.80-1.85 (1H, m), 2.03-2.10 (1H, m), 3.06 (1H, d, J=4.4 Hz), 3.89 (3H, s), 4.35 (2H, q, J=7.6 Hz).

### Preparation of (1,7,8,8-tetramethyl-4,5,6,7-tetrahydro-1H-4,7-methanoindazol-3-yl)methanol (6)

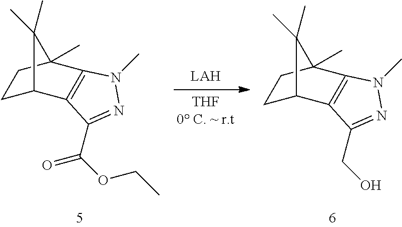

To a solution of ethyl 1,7,8,8-tetramethyl-4,5,6,7-tetrahydro-1H-4,7 methanoindazole-3-carboxylate 5 (1.6 g, 6.09 mmol) in THF (20 mT) at 0° C. was added a solution of 1M lithium aluminum hydride in THF (9.1 mL, 9.1 mmol) dropwise via a syringe. The resulting mixture was stirred at 0° C. to room temperature overnight, poured into saturated NH₄Cl aqueous solution (200 mL) slowly and extracted with EtOAc (3×20 mL). The combined EtOAc layers were washed with water, brine, dried over Na₂SO₄ and concentrated under reduced pressure to give crude 6 (1.5 g, quant. yield) as a pale yellow oil which was used without further purification.

¹H NMR (400 MHz, CDCl₃): δ 0.75 (3H, s), 0.89 (3H, s), 1.04-1.10 (1H, m), 1.15-1.20 (1H, m), 1.33 (3H, s), 1.75-1.78 (1H, m), 1.99-2.06 (1H, m), 2.65 (1H, br s), 2.78 (1H, d, J=4.0 Hz), 3.78 (3H, s), 4.59 (2H, s).

### Preparation of 1,7,8,8-tetramethyl-4,5,6,7-tetrahydro-1H-4,7-methanoindazole-3-carbaldehyde (7)

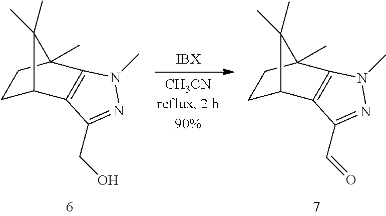

To a solution of (1,7,8,8-tetramethyl-4,5,6,7-tetrahydro-1H-4,7-methanoindazol-3-yl)methanol 6 (1.5 g, 6.09 mmol) in acetonitrile (50 mL) was added 1-hydroxy-1,2-benziodoxol-3H-one-1-oxide (2.04 g, 7.31 mmol). The resulting mixture was heated under reflux for 2 h and TLC indicated the reaction was complete. After cooling to room temperature, the reaction mixture was filtered and washed with acetonitrile. The filtrate was concentrated under reduced pressure. The residue was purified by column chromatography (silica gel, hexane:EtOAc, 3:1) to afford 7 (1.2 g, 90%, two steps) as a pale yellow solid.

¹H NMR (400 MHz, CDCl₃): δ 0.74 (3H, s), 0.92 (3H, s), 1.04-1.10 (1H, m), 1.14-1.20 (1H, m), 1.37 (3H, s), 1.81-1.88 (1H, m), 2.04-2.12 (1H, m), 3.10 (1H, d, J=4.0 Hz), 3.93 (3H, s), 9.92 (1H, s).

### Preparation of 4-nitrobenzyl (5R)-6-[(acetyloxy)(1,7,8,8-tetramethyl-4,5,6,7-tetrahydro-1H-4,7-methanoindazol-3-yl)methyl]-6-bromo-7-oxo-4-thia-1-azabicyclo[3.2.0]hept-2-ene-2-carboxylate (9)

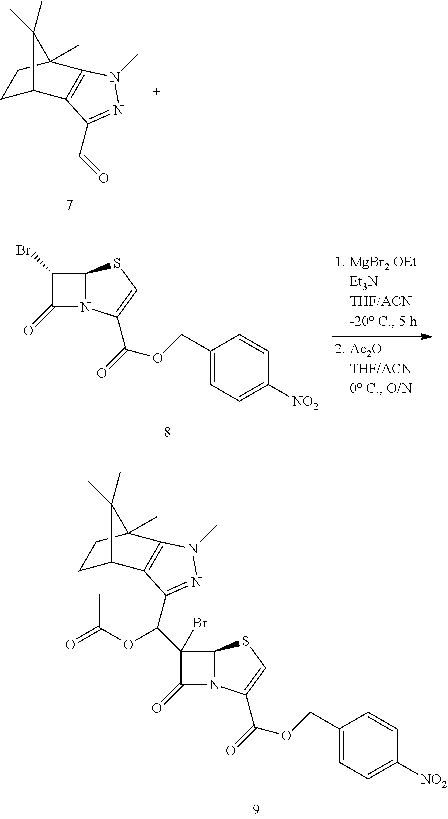

To a mixture of 1,7,8,8-tetramethyl-4,5,6,7-tetrahydro-1H-4,7-methanoindazole-3-carbaldehyde 7 (0.32 g, 1.46 mmol), 4-nitrobenzyl (5R,6S)-6-bromo-7-oxo-4-thia-1-azabicyclo[3.2.0]hept-2-ene-2-carboxylate 8 (0.56 g, 1.46 mmol) and magnesium bromide diethyl etherate (0.75 g, 2.92 mmol) in THF (10 mL) and acetonitrile (15 mL) at −20° C. under nitrogen was added Et₃N (0.40 mL, 2.92 mmol). The resulting mixture was stirred at −20° C. for 5 h while the reaction flask was covered by aluminum foil to exclude light, and then acetic anhydride (0.69 mL, 7.3 mmol) was added slowly via a syringe. The resulting mixture was stirred at 0° C. overnight and diluted with EtOAc (100 mL), washed with 5% citric acid, saturated NaHCO₃ aqueous solution, brine, dried over Na₂SO₄ and concentrated under reduced pressure. The residue was purified by column chromatography (silica gel, hexane:EtOAc, 6:1 to 4:1) to afford 9 (0.39 g, 41%) as a yellow solid as a mixture of diastereoisomers.

¹H NMR (400 MHz, CDCl₃): δ 0.70-0.79 (3H, m), 0.86-0.92 (3H, s), 0.94-1.05 (1H, m), 1.13-1.30 (1H, m), 1.32 (3H, s), 1.78-1.82 (1H, m), 1.91-2.09 (1H, m), 2.24 (3H, s), 2.60-2.77 (1H, m), 3.77 (3H, s), 5.27 (1H, d, J=13.7 Hz, ABq), 5.44 (1H, d, J=13.7 Hz, ABq), 5.96 (0.5H, s), 6.02 (0.5H, s), 6.69 (0.5H, s), 6.78 (0.5H, s), 7.43 (0.5H, s), 7.45 (0.5H, s), 7.61 (2H, d, J=8.60 Hz), 8.24 (2H, d, J=8.60 Hz).

### Preparation of (5R,6Z)-7-oxo-6-[(1,7,8,8-tetramethyl-4,5,6,7-tetrahydro-1H-4,7-methanoindazol-3-yl)methylidene]-4-thia-1-azabicyclo[3.2.0]hept-2-ene-2-carboxylic acid (compound A)

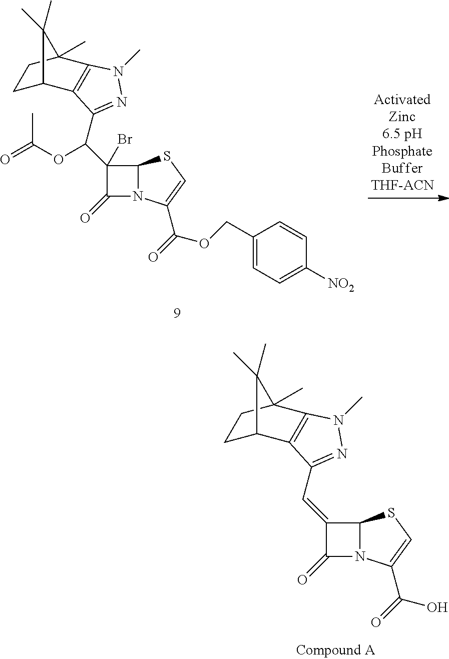

To a suspension of activated Zinc dust (10 g in THF, activated by washing with 0.1 N HCl, water and THF, and kept in THF) in THF (10 mL) and acetonitrile (5 ml) at room temperature was added 4-nitrobenzyl (5R)-6-[(acetyloxy)(1,7,8,8-tetramethyl-4,5,6,7-tetrahydro-1H-4,7-methanoindazol-3-yl)methyl]-6-bromo-7-oxo-4-thia-1-azabicyclo[3.2.0]hept-2-ene-2-carboxylate 9 (0.39 g, 0.60 mmol) followed by 0.5 M phosphate buffer (pH 6.5, 30 mL). The resulting mixture was vigorously stirred at room temperature for 4 h while the reaction flask was covered by aluminum foil to exclude light. After 4 h the reaction mixture was filtered. The filtrate was washed with EtOAc (2×15 mL) and the aqueous layer was separated. The aqueous layer was loaded over SP-207 resin reverse phase column (12 cm×3 cm) and eluted with water (1 L) and with 20% acetonitrile in water. The fractions containing product were collected and concentrated under reduced pressure at room temperature to remove organic solvent and freeze-dried to give the product (0.17 g, 72%) as sodium salt (100 mg), which was further purified by preparative HPLC to provide Compound A (50 mg) as a mixture of diastereoisomers

¹H NMR (400 MHz, CDCl₃): δ 0.75-0.76 (3H, m), 0.92-0.96 (3H, m), 1.02-1.11 (1H, m), 1.16-1.20 (1H, m), 1.35 (3H, s), 1.80-1.86 (1H, m), 2.04-2.10 (1H, m), 2.79-2.82 (1H, m), 3.76 (1H, br s), 3.87 (3H, s), 6.45 (1H, s), 7.02 (1H, s), 7.36 (1H, m).

HPLC purity: 92.45% (51.60+40.85%, 2 isomers).

MS (ES⁺) m/z: [M+H]⁺ calcd for C₁₉H₂₂O₃N₃S: 372.14. Found: 372.26.

### Example 2

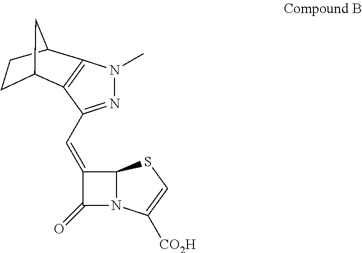

### Preparation of ethyl 2-hydroxy(3-oxobicyclo[2.2.1]hept-2-ylidene)ethanoate (3)

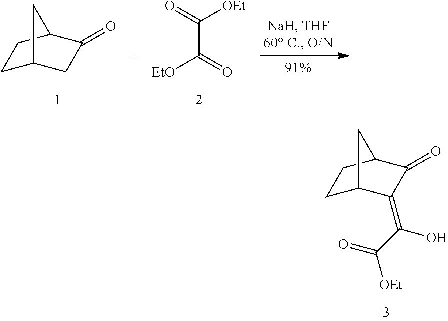

To a mixture of bicyclo[2.2.1]heptan-2-one 1 (5.00 g, 45.5 mmol) and diethyl ethanedioate 2 (7.40 mL, 54.5 mmol) in THF (100 mL) was added sodium hydride (2.36 g, 59.1 mmol) in portions. The mixture was stirred at 60° C. overnight, quenched with ice-water, acidified with 2N HCl aqueous solution, extracted with EtOAc and concentrated to provide a residue, which was subjected to chromatography (silica gel, hexanes:EtOAc, 7:1) to give 3 (8.71 g, 91%) as a colorless oil.

¹H NMR (400 MHz, CDCl₃): δ1.38 (3H, t, J=7.2 Hz), 1.68 (3H, m), 1.81 (1H, d, J=10.4 Hz), 1.96 (2H, m), 2.80 (1H, s), 3.80 (1H, s), 4.34 (2H, q, J=6.8 Hz), 11.35 (1H, br s).

### Preparation of ethyl 1-methyl-4,5,6,7-tetrahydro-1H-4,7-methanoindazole-3-carboxylate (4) and ethyl 2-methyl-4,5,6,7-tetrahydro-2H-4,7-methanoindazole-3-carboxylate (5)

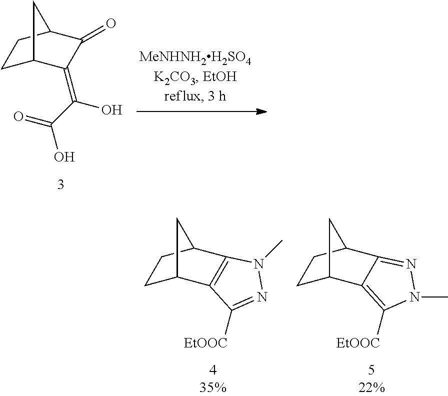

A mixture of ethyl 2-hydroxy(3-oxobicyclo[2.2.1]hept-2-ylidene)ethanoate 3 (3.00 g, 14.3 mmol), methylhydrazine sulfate (4.11 g, 28.5 mmol) and K₂CO₃ (3.84 g, 28.5 mmol) in EtOH (200 mL) was refluxed for 3 h and concentrated to provide a white solid, which was diluted with water and EtOAc. The organic layer was separated and concentrated to give a residue, which was subjected to chromatography (silica gel, hexanes:EtOAc, 4:1 to 2:1) to provide 4 (1.08 g, 35%) as a yellow oil and 5 (0.762 g, 22%) as a colorless oil.

Compound 4:

¹H NMR (400 MHz, CDCl₃): δ 1.10 (2H, m), 1.39 (3H, t, J=7.2 Hz), 1.65 (1H, d, J=8.4 Hz), 1.91 (2H, m), 2.01 (1H, d, J=8.4 Hz), 3.39 (1H, s), 3.60 (1H, s), 3.87 (3H, s), 4.36 (2H, q, J=7.2 Hz).

Compound 5:

¹H NMR (400 MHz, CDCl₃): δ 1.11 (2H, m), 1.39 (3H, m), 1.65 (1H, m), 1.95 (3H, m), 3.40 (1H, s), 3.55 (1H, s), 4.08 (3H, s), 4.36 (2H, m).

### Preparation (1-methyl-4,5,6,7-tetrahydro-1H-4,7-methanoindazol-3-yl)methanol (6)

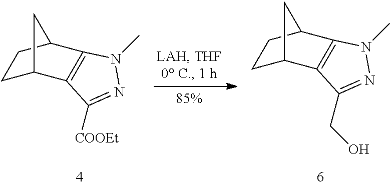

To a mixture of ethyl 1-methyl-4,5,6,7-tetrahydro-1H-4,7-methanoindazole-3-carboxylate 4 (1.08 g, 4.91 mmol) in THF (20 mL) was added lithium aluminum hydride (1M in THF, 7.36 mL, 7.36 mmol) at 0° C. The reaction mixture was stirred at 0° C. for 1 h, quenched with NaOH aqueous solution, extracted with EtOAc and concentrated to provide 6 (0.74 g, 85%) as a colorless oil. The product was used in the next step without further purification.

### Preparation of 1-methyl-4,5,6,7-tetrahydro-1H-4,7-methanoindazole-3-carbaldehyde (7)

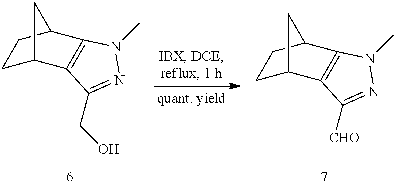

A mixture of (1-methyl-4,5,6,7-tetrahydro-1H-4,7-methanoindazol-3-yl)methanol 6 (0.740 g, 4.15 mmol) and 1-hydroxy-1,2-benziodoxol-3H-one-1-oxide (1.75 g, 6.22 mmol) in dichloroethane (20 mL) was refluxed for 1 h, filtered through a pad of celite, washed with dichloromethane and concentrated to provide a residue, which was subjected to chromatography (silica gel, hexanes:EtOAc, 4:1 to 3:1) to give 7 (0.82 g, quant. yield) as a colorless oil.

¹H NMR (400 MHz, CDCl₃): δ 1.09 (2H, m), 1.66 (1H, m), 1.92 (2H, m), 2.02 (1H, m), 3.41 (1H, s), 3.66 (1H, s), 3.91 (3H, s), 9.88 (1H, s).

### Preparation of 4-nitrobenzyl (5R)-6-[(acetyloxy)(1-methyl-4,5,6,7-tetrahydro-1H-4,7-methanoindazol-3-yl)methyl]-6-bromo-7-oxo-4-thia-1-azabicyclo[3.2.0]hept-2-ene-2-carboxylate (9)

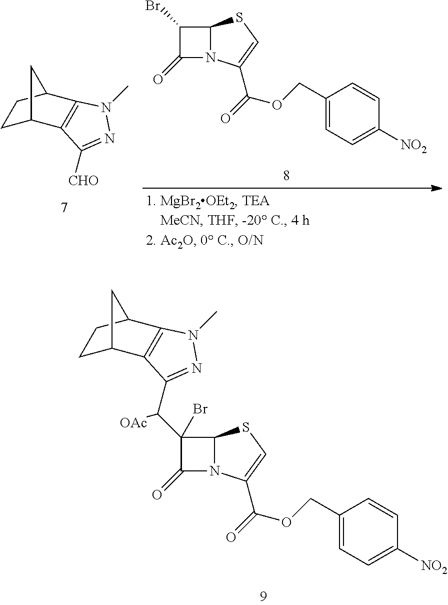

To a mixture of 1-methyl-4,5,6,7-tetrahydro-1H-4,7-methanoindazole-3-carbaldehyde 7 (0.152 g, 0.865 mmol), 4-nitrobenzyl (5R,6S)-6-bromo-7-oxo-4-thia-1-azabicyclo[3.2.0]hept-2-ene-2-carboxylate 8 (0.400 g, 1.04 mmol) and magnesium bromide diethyl etherate (0.446 g, 1.73 mmol) in a solution of THF (15 mL) and acetonitrile (10 mL) was added Et₃N (0.241 mL, 1.73 mmol) at −20° C. under nitrogen. The resulting mixture was stirred at −20° C. for 4 h while the reaction flask was covered by aluminum foil to exclude light, and then acetic anhydride (0.408 mL, 4.32 mmol) was added. The resulting mixture was kept at 0° C. overnight and diluted with EtOAc, washed with 10% citric acid, sat. NaHCO₃ aqueous solution, brine, dried over Na₂SO₄ and concentrated to provide a residue, which was subjected to chromatography (silica gel, hexanes:EtOAc, 4:1 to 2:1) to give 9 as a mixture of diastereoisomers in 50% yield.

Mixture 1:

¹H NMR (400 MHz, CDCl₃): δ 1.10 (3H, m), 1.61 (1H, t, J=11.2 Hz), 2.00 (2H, m), 2.28 (3H, m), 3.28 (2H, m), 3.75 (3H, m), 5.28 (1H, d, J=13.6 Hz), 5.44 (1H, d, J=13.6 Hz), 6.00-6.82 (2H, m), 7.43 (1H, m), 7.62 (2H, d, J=6.8 Hz), 8.26 (2H, d, J=6.8 Hz).

Mixture 2:

¹H NMR (400 MHz, CDCl₃): δ 1.10 (3H, m), 1.61 (1H, m), 2.00 (2H, m), 2.18 (3H, m), 3.41 (2H, m), 3.78 (3H, m), 5.29 (1H, d, J=11.2 Hz), 5.51 (1H, d, J=11.2 Hz), 6.22 (1H, m), 6.42 (1H, m), 7.45 (1H, m), 7.62 (2H, d, J=5.6 Hz), 8.26 (2H, d, J=5.6 Hz).

### Preparation of (5R,6Z)-6-[(1-methyl-4,5,6,7-tetrahydro-1H-4,7-methanoindazol-3-yl)methylidene]-7-oxo-4-thia-1-azabicyclo[3.2.0]hept-2-ene-2-carboxylic acid (Compound B)

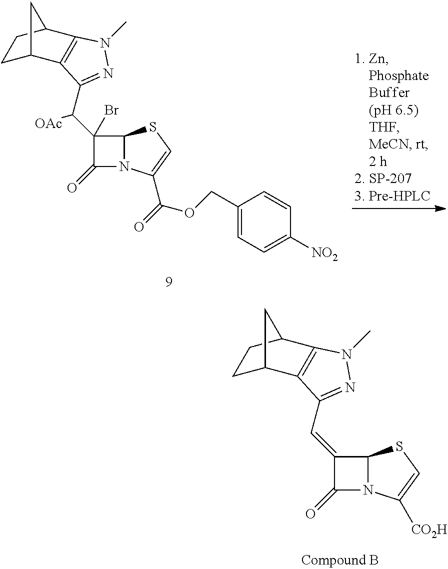

To a mixture of 4-nitrobenzyl (5R)-6-[(acetyloxy)(1-methyl-4,5,6,7-tetrahydro-1H-4,7-methanoindazol-3-yl)methyl]-6-bromo-7-oxo-4-thia-1-azabicyclo[3.2.0]hept-2-ene-2-carboxylate 9 (0.15 g, 0.25 mmol) in MeCN (1.5 mL) and THF (3.0 mL) was added activated Zinc dust (2 g wet, activated by washing with 0.1 N HCl and water) followed by 0.5 M phosphate buffer (pH 6.5, 5.0 mL). The resulting mixture was vigorously stirred at room temperature for 2 h. The reaction flask was covered by aluminum foil to exclude light. After 2 h the reaction mixture was filtered through a pad of Celite. The filtrate was washed with EtOAc and the aqueous layer was separated. The aqueous layer was loaded over SP-207 resin column and eluted with water (750 mL) and then with 20% acetonitrile in water. The yellow fractions were collected and concentrated under reduced pressure at 30° C. to remove organic solvent and freeze-dried to give product (27 mg, 31%) as sodium salt, which was further purified by preparative HPLC to give Compound B (7 mg) as a yellow solid.

¹H NMR (400 MHz, CDCl₃): δ 1.11 (2H, m), 1.66 (1H, m), 1.99 (3H, m), 3.39 (2H, m), 3.84 (3H, s), 6.46 (1H, m), 7.02 (1H, s), 7.34 (1H, s).

HPLC purity: 86.82%.

MS (ES⁺) m/z: [M+H]⁺ calcd for C₁₆H₁₆N₃O₃S: 330.09. Found: 330.17.

### Example 3

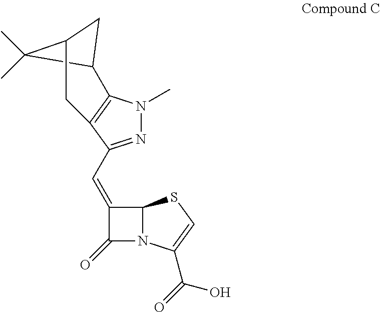

### Preparation of ethyl (2Z)-(6,6-dimethyl-2-oxobicyclo[3.1.1]hept-3-ylidene)(hydroxy)ethanoate (2)

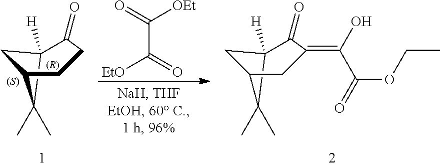

A suspension of diethyl ethanedioate (3.60 mL, 26.5 mmol) and sodium hydride (0.78 g, 32.5 mmol) in tetrahydrofuran (100 mL) was heated at 60° C. for 10 minutes. A solution of 6,6-dimethylbicyclo[3.1.1]heptan-2-one 1 ((1R)-(+) nopinone, 3.00 g, 21.7 mmol) in tetrahydrofuran (5 mL) was added to the mixture followed by ethanol (0.1 mL). After the effervescence subsided, the mixture was heated for 1 hour at 60° C., then cooled to room temperature, diluted with ice-cold water, acidified with 2N aqueous hydrochloric acid, extracted with ethyl acetate, dried (Na₂SO₄), filtered and concentrated in vacuo to a yellow oil. The oil was purified by chromatography (hexanes:ethyl acetate, 7:1) to afford 2 (5.00 g, 96%) as a light yellow oil.

¹H NMR (400 MHz, CDCl₃): δ 0.92 (3H, s), 1.36 (3H, s), 1.40 (3H, t, J=7.02 Hz), 1.44 (1H, d, J=9.15 Hz), 2.31 (1H, m), 2.59 (2H, m), 2.89 (2H, m), 4.36 (2H, q, J=7.02 Hz), 14.38 (1H, s).

### Preparation of ethyl 1,6,6-trimethyl-4,5,6,7-tetrahydro-1H-5,7-methanoindazole-3-carboxylate (3) and ethyl 2,6,6-trimethyl-4,5,6,7-tetrahydro-2H-5,7-methanoindazole-3-carboxylate (4)

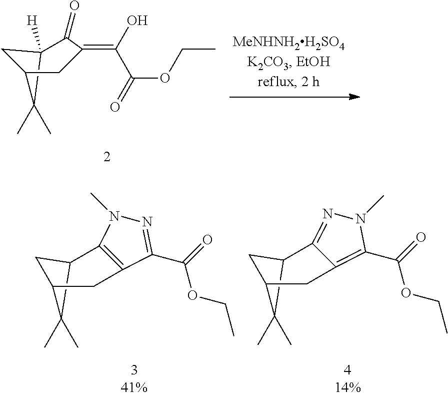

A mixture of ethyl (2Z)-(6,6-dimethyl-2-oxobicyclo[3.1.1]hept-3-ylidene)(hydroxy)ethanoate 2 (0.14 g, 0.59 mmol), methylhydrazine sulfate (0.17 g, 1.17 mmol) and potassium carbonate (0.16 g, 1.17 mmol) in ethanol (10 mL) was refluxed for 2 hours, cooled to room temperature, then concentrated to provide a white solid, which was diluted with water and ethyl acetate. The organic extract was dried over Na₂SO₄, filtered and concentrated in vacuo to a brown oil. The oil was purified by chromatography (silica gel, hexanes:ethyl acetate, 7:1 to 1:1) to provide 3 (0.06 g, 41%) as a yellow solid and 4 (0.02 g, 14%) as a yellow oil.

Compound 3:

¹H NMR (400 MHz, CDCl₃): δ 0.66 (3H, s), 1.35 (1H, d, J=9.38 Hz), 1.40 (3H, t, J=7.03 Hz), 1.42 (3H, s), 2.31 (1H, m), 2.72 (1H, m), 2.89 (3H, m), 3.79 (3H, s), 4.39 (2H, q, J=7.03 Hz).

Compound 4:

¹H NMR (400 MHz, CDCl₃): δ 0.67 (3H, s), 1.30 (1H, d, J=9.77 Hz), 1.38 (3H, t, J=7.03 Hz), 1.40 (3H, s), 2.26 (1H, m), 2.71 (1H, m), 2.85 (3H, m), 4.08 (3H, s), 4.33 (2H, m).

### Preparation of (1,8,8-Trimethyl-4,5,6,7-tetrahydro-1H-5,7-methano-indazol-3-yl)-methanol (5)

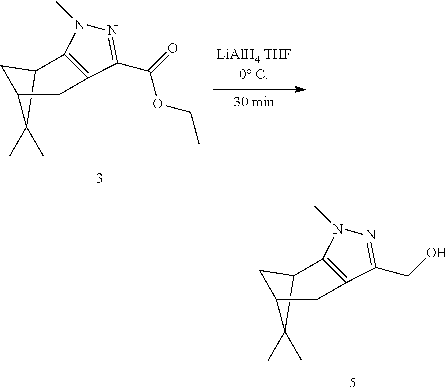

To a mixture of ethyl 1,6,6-trimethyl-4,5,6,7-tetrahydro-1H-5,7-methanoindazole-3-carboxylate 3 (0.50 g, 2.01 mmol) in tetrahydrofuran (20 mL) was added lithium aluminum hydride (1M in THF, 4.00 mL, 4.00 mmol) at 0° C. The reaction mixture was stirred at 0° C. for 30 minutes, quenched with saturated ammonium chloride, dried over Na₂SO₄, filtered through a Celite pad and the filtrate was concentrated in vacuo to a light yellow oil to afford 5 which was used in the next step without further purification.

¹H NMR (400 MHz, CDCl₃): δ 0.67 (3H, s), 1.36 (1H, d, J=9.38 Hz), 1.41 (3H, s), 2.14 (1H, br s), 2.29 (1H, m), 2.68 (3H, m), 2.80 (1H, t, J=5.08 Hz), 3.69 (3H, s), 4.63 (2H, s).

### Preparation of 1,6,6-trimethyl-4,5,6,7-tetrahydro-1H-5,7-methanoindazole-3-carbaldehyde (6)

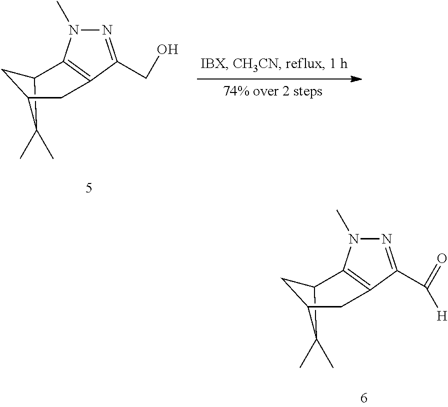

A mixture of (1,8,8-trimethyl-4,5,6,7-tetrahydro-1H-5,7-methano-indazol-3-yl)-methanol (0.42 g, 2.01 mmol) and 1-hydroxy-1,2-benziodoxol-3H-one-1-oxide (0.82 g, 2.93 mmol) in acetonitrile (10 mL) was refluxed for 1 hour. The suspension was filtered through a pad of Celite and the filtrate was concentrated to a yellow oil. The oil was purified by chromatography (silica gel, hexanes:ethyl acetate, 3:1 to 1:1) to afford 6 (0.30 g, 74% over 2 steps) as a yellow oil.

¹H NMR (400 MHz, CDCl₃): δ 0.66 (3H, s), 1.35 (1H, d, J=9.38 Hz), 1.44 (3H, s), 2.32 (1H, m), 2.74 (1H, m), 2.91 (3H, m), 3.82 (3H, s), 9.96 (1H, s).

### Preparation of 4-nitrobenzyl (5R)-6-[(acetyloxy)(1,6,6-trimethyl-4,5,6,7-tetrahydro-1H-5,7-methanoindazol-3-yl)methyl]-6-bromo-7-oxo-4-thia-1-azabicyclo[3.2.0]hept-2-ene-2-carboxylate (8)

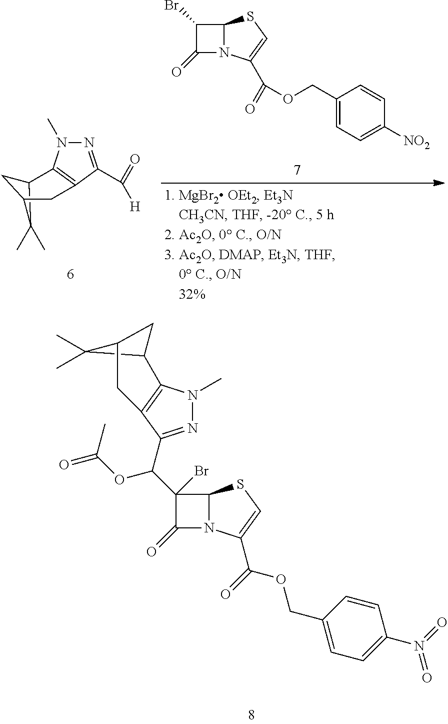

To a mixture of 1,6,6-trimethyl-4,5,6,7-tetrahydro-1H-5,7-methanoindazole-3-carbaldehyde 6 (0.305 g, 0.1.50 mmol), 4-nitrobenzyl (5R,6S)-6-bromo-7-oxo-4-thia-1-azabicyclo[3.2.0]hept-2-ene-2-carboxylate 7 (0.63 g, 1.63 mmol) and magnesium bromide diethyl etherate (0.1.15 g, 4.45 mmol) in a mixture of tetrahydrofuran (12 mL) and acetonitrile (12 mL) was added triethylamine (1.00 mL, 7.17 mmol) at −20° C. under nitrogen. The resulting mixture was stirred at −20° C. for 5 hours while the reaction flask was covered with aluminum foil to exclude light, then acetic anhydride (0.30 mL, 3.17 mmol) was added. The resulting mixture was kept at 0° C. overnight, diluted with ethyl acetate and washed with water. The organic extract was dried over Na₂SO₄ and concentrated to provide a brown foam. The foam was purified by chromatography (silica gel, hexanes:ethyl acetate, 4:1 to 1:1) to afford mixture 1 (0.33 g) followed by mixture 2 (0.10 g). Both the mixtures were a mixture of 8 and its hydroxyl analog based on mass spectroscopy. Mixture 1 (330 mg) was diluted with tetrahydrofuran (10 then treated with triethylamine (0.8 mL, 5.74 mmol) followed by acetic anhydride at −20° C. The flask was covered with aluminum foil to exclude light. After the mixture was stirred for 2 hours, 4-(dimethylamino)pyridine (0.02 g, 0.16 mmol) was added and the mixture was stirred at 0° C. overnight. The mixture was diluted with water and extracted with dichloromethane. The organic extract was dried over Na₂SO₄ and concentrated to provide a yellow foam, which was purified by chromatography (silica gel, hexanes:ethyl acetate, 3:1 to 1:1) to afford 8 (0.30 g, 32%).

¹H NMR (400 MHz, CDCl₃): δ 0.64 (0.8H, s), 0.71 (2.2H, s), 1.33 (1H, m), 2.03 (1H, m), 2.26 (3H, s), 2.62 (2H, m), 2.78 (2H, m), 3.65 (3H, s), 3.70 (3H, s), 5.26 (1H, m), 5.45 (1H, m), 6.04 (0.6H, s), 6.25 (0.4H, s), 6.39 (0.4H, s), 6.91 (0.6H, s), 7.45 (0.6H, s), 7.49 (0.4H, s), 7.62 (2H, m), 8.24 (2H, d, J=8.60 Hz).

### Preparation of (5R,6Z)-7-oxo-6-[(1,6,6-trimethyl-4,5,6,7-tetrahydro-1H-5,7-methanoindazol-3-yl)methylidene]-4-thia-1-azabicyclo[3.2.0]hept-2-ene-2-carboxylic acid (Compound C)

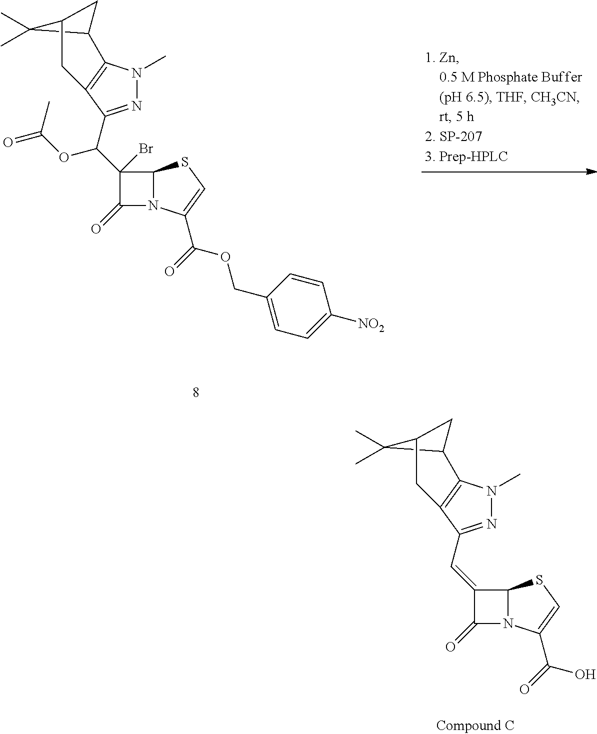

To a mixture of 4-nitrobenzyl (5R)-6-[(acetyloxy)(1-methyl-4,5,6,7-tetrahydro-1H-4,7-methanoindazol-3-yl)methyl]-6-bromo-7-oxo-4-thia-1-azabicyclo[3.2.0]hept-2-ene-2-carboxylate 8 (0.10 g, 0.16 mmol) in a solution of acetonitrile (2.0 mL) and tetrahydrofuran (4.0 mL) was added activated zinc dust (0.60 g wet, activated by washing with 0.1 N HCl and water) followed by 0.5 M phosphate buffer (pH 6.5, 3.0 mL). The resulting mixture was vigorously stirred at room temperature for 7 hours. The reaction flask was covered by aluminum foil to exclude light. After 7 hours the reaction mixture was filtered through a pad of Celite. The filtrate was washed with EtOAc and the aqueous layer was separated. The aqueous layer was loaded over SP-207 resin column and eluted with water followed by 20% acetonitrile in water. The yellow fractions were collected and concentrated under reduced pressure at 30° C. to remove organic solvent and lyophilized to afford product (30 mg, 53%) as a sodium salt, which was further purified by preparative HPLC to afford Compound C (13 mg) as a yellow solid.

¹H NMR (400 MHz, CDCl₃): δ 0.67 (3H, s), 1.34 (1H, d, J=9.38 Hz), 1.43 (3H, s), 2.31 (1H, m), 2.70 (3H, m), 2.83 (1H, m), 3.75 (3H, s), 6.49 (1H, s), 7.00 (1H, s), 7.31 (1H, s).

HPLC purity: 86.75%.

MS (ES⁺) m/z: [M+H]⁺ calcd for C₁₈H₂₀N₃O₃S: 358.12. Found: 358.17.

### Example 4

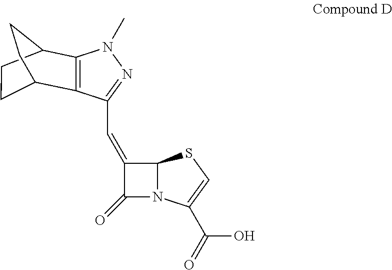

### Preparation of 5-cyanobicyclo[2.2.2]oct-2-ene (3)

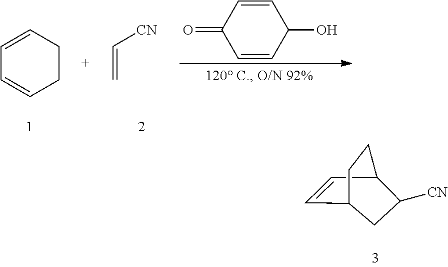

A mixture of 1,3-cyclohexadiene 1 (6.00 g, 74.9 mmol), acrylonitrile 2 (9.84 mL, 149 mmol) and hydroquinone (0.102 g, 0.936 mmol) in a sealed tube was heated at 120° C. overnight. The mixture was concentrated and washed with hexanes to provide 3 (9.31 g, 92%) as a sticky gum.

¹H NMR (400 MHz, CDCl₃): δ 1.22-1.77 (5H, m), 1.92-2.09 (1H, m), 2.41-2.92 (3H, m), 6.22-6.50 (2H, m).

### Preparation of 5-chloro-5-cyanobicyclo[2.2.2]oct-2-ene (4)

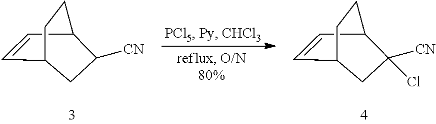

To a mixture of phosphorus pentachloride (24.5 g, 103 mmol) in chloroform (60 mL) was added pyridine (11.1 mL, 137 mmol) slowly. The mixture was refluxed. A solution of 5-cyanobicyclo[2.2.2]oct-2-ene 3 (9.30 g, 68.6 mmol) was added to above mixture. The resulting mixture was refluxed overnight, cooled to room temperature, quenched with water, extracted with EtOAc and concentrated to provide 4 (9.25 g, 80%) as a yellow solid.

¹H NMR (400 MHz, CDCl₃): δ 1.26-1.70 (3H, m), 2.01-2.54 (3H, m), 2.71-2.75 (1H, m), 3.04-3.15 (1H, m), 6.22-6.53 (2H, m).

### Preparation of bicyclo[2.2.2]oct-5-en-2-one (5)

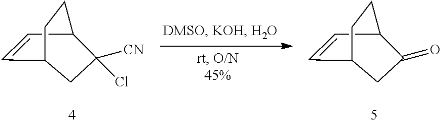

To a mixture of 5-chloro-5-cyanobicyclo[2.2.2]oct-2-ene 4 (2.50 g, 14.9 mmol) in dimethylsulfoxide (20 mL) was added a solution of KOH (3.34 g, 59.6 mmol) in water (2 mL) slowly. The mixture was vigorously stirred overnight at room temperature, quenched with water, extracted with EtOAc and concentrated to provide a residue, which was subjected to chromatography (silica gel, hexanes:EtOAc, 5:1) to give 5 (0.82 g, 45%) as a white solid.

¹H NMR (400 MHz, CDCl₃): δ 1.55 (2H, m), 1.59 (1H, m), 1.86 (1H, m), 2.04 (2H, s), 2.99 (1H, m), 3.13 (1H, m), 6.21 (1H, t, J=7.6 Hz), 6.48 (1H, t, J=7.6 Hz).

### Preparation of bicyclo[2.2.2]octan-2-ol (6)

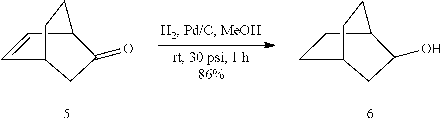

A mixture of bicyclo[2.2.2]oct-5-en-2-one 5 (0.820 g, 6.56 mmol) and Pd/C (5%, 0.30 g) in MeOH (100 mL) was hydrogenated under 30 psi at room temperature for 1 h. The mixture was filtered through a pad of celite and concentrated to provide 6 (0.71 g, 86%) as a colorless oil.

¹H NMR (400 MHz, CDCl₃): δ 1.61 (2H, m), 1.71 (2H, m), 1.82 (4H, m), 2.15 (1H, m), 2.25 (3H, m), 3.49 (1H, d, J=4.8 Hz).

### Preparation of bicyclo[2.2.2]octan-2-one (7)

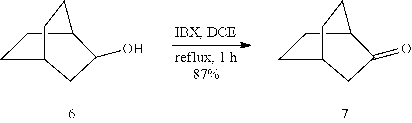

A mixture of bicyclo[2.2.2]octan-2-ol 6 (0.710 g, 5.72 mmol) and 1-hydroxy-1,2-benziodoxol-3H-one-1-oxide (2.40 g, 8.59 mmol) in dichloroethane (20 mL) was refluxed for 1 h, filtered through a pad of celite, washed with dichloromethane and concentrated to provide 7 (0.62 g, 87%) as a white solid.

¹H NMR (400 MHz, CDCl₃): δ 1.62 (2H, m), 1.70 (2H, m), 1.82 (4H, m), 2.15 (1H, m), 2.25 (3H, m).

### Preparation of ethyl hydroxy(3-oxobicyclo[2.2.2]oct-2-ylidene)acetate (8)

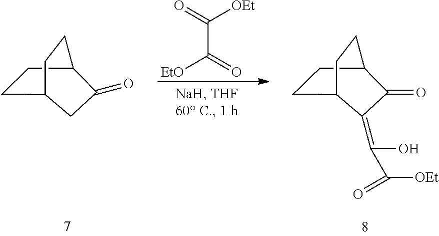

To a mixture of bicyclo[2.2.2]octan-2-one 7 (2.22 g, 17.9 mmol) and diethyl oxalate (2.91 mL, 21.5 mmol) in THF (60 mL) was added NaH (0.930 g, 23.3 mmol) in portions followed by few drops of EtOH. The mixture was stirred at 60° C. for 1 h, quenched with ice-water, acidified with 2N HCl aqueous solution, extracted with EtOAc and concentrated to provide crude 8 (4.84 g) as a brown oil, which was used in the next reaction without further purification.

¹H NMR (400 MHz, CDCl₃): δ 1.38 (3H, m), 1.62 (1H, m), 1.78 (6H, m), 2.52 (1H, s), 3.57 (1H, s), 4.35 (2H, m), 13.80 (1H, br s).

### Preparation of ethyl 1-methyl-4,5,6,7-tetrahydro-1H-4,7-ethanoindazole-3-carboxylate (9) and ethyl 2-methyl-4,5,6,7-tetrahydro-2H-4,7-ethanoindazole-3-carboxylate (10)

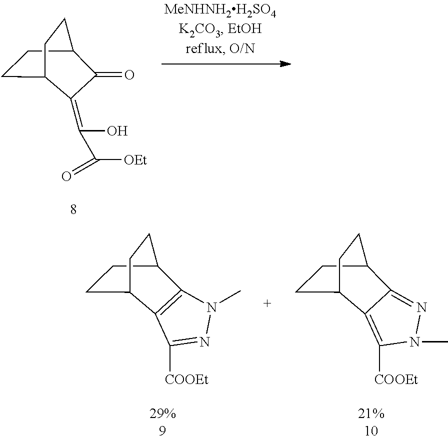

A mixture of ethyl hydroxy(3-oxobicyclo[2.2.2]oct-2-ylidene)acetate 8 (4.84 g, crude), methyl hydrazine sulfate (5.16 g, 35.8 mmol) and K₂CO₃ (4.94 g, 35.8 mmol) in EtOH (200 mL) was refluxed overnight and concentrated to provide a white solid, which was diluted with water and EtOAc. The organic layer was separated and concentrated to give a residue, which was subjected to chromatography (silica gel, hexanes:EtOAc, 4:1 to 2:1) to provide 9 (1.20 g, 29%) as a yellow oil and 10 (0.900 g, 21%) as a white solid.

Compound 9:

¹H NMR (400 MHz, CDCl₃): δ 1.32 (4H, d, J=8.0 Hz), 1.40 (3H, t, J=7.2 Hz), 1.76 (4H, m), 3.22 (1H, s), 3.62 (1H, s), 3.91 (3H, s), 4.39 (2H, q, J=7.2 Hz).

Compound 10:

¹H NMR (400 MHz, CDCl₃): δ 1.38 (7H, m), 1.78 (4H, m), 3.15 (1H, m), 3.46 (1H, m), 4.13 (3H, s), 4.35 (2H, q, J=8.0 Hz).

### Preparation of (1-methyl-4,5,6,7-tetrahydro-1H-4,7-ethanoindazol-3-yl)methanol (11)

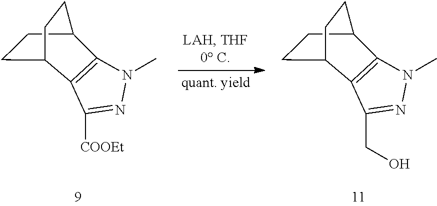

To a mixture of ethyl 1-methyl-4,5,6,7-tetrahydro-1H-4,7-ethanoindazole-3-carboxylate 9 (1.20 g, 5.12 mmol) in THF (40 mL) was added lithium aluminum hydride (1M in THF, 5.12 mL, 5.12 mmol) at 0° C. The reaction mixture was stirred at 0° C. for 1 h, quenched with NaOH aqueous solution, extracted with EtOAc and concentrated to provide 11 (1.0 g, quant. yield) as a yellow oil. The product was used in the next step without further purification.

¹H NMR (400 MHz, CDCl₃): δ 1.26 (4H, m), 1.74 (4H, m), 3.18 (2H, m), 3.81 (3H, s), 4.68 (2H, d, J=5.6 Hz).

### Preparation of 1-methyl-4,5,6,7-tetrahydro-1H-4,7-ethanoindazole-3-carbaldehyde (12)

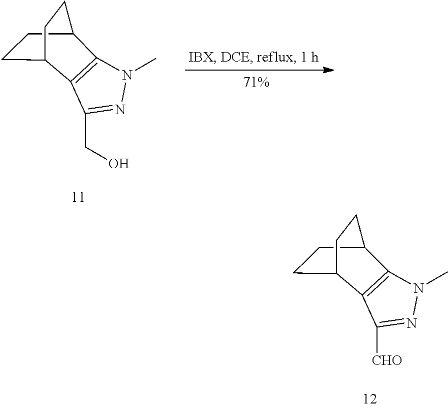

A mixture of (1-methyl-4,5,6,7-tetrahydro-1H-4,7-ethanoindazol-3-yl)methanol 11 (1.00 g, 5.20 mmol) and 1-hydroxy-1,2-benziodoxol-3H-one-1-oxide (2.18 g, 7.80 mmol) in dichloroethane (40 mL) was refluxed for 1 h, filtered through a pad of celite, washed with dichloromethane and concentrated to provide a residue, which was subjected to chromatography (silica gel, hexanes:EtOAc, 4:1 to 3:1) to give 12 (0.70 g, 71%) as a yellow oil.

¹H NMR (400 MHz, CDCl₃): δ 1.29 (4H, m), 1.78 (4H, m), 3.24 (1H, s), 3.66 (1H, s), 3.94 (3H, s), 9.99 (1H, s).

### Preparation of 4-nitrobenzyl (5R)-6-[(acetyloxy)(1-methyl-4,5,6,7-tetrahydro-1H-4,7-ethanoindazol-3-yl)methyl]-6-bromo-7-oxo-4-thia-1-azabicyclo[3.2.0]hept-2-ene-2-carboxylate (14)

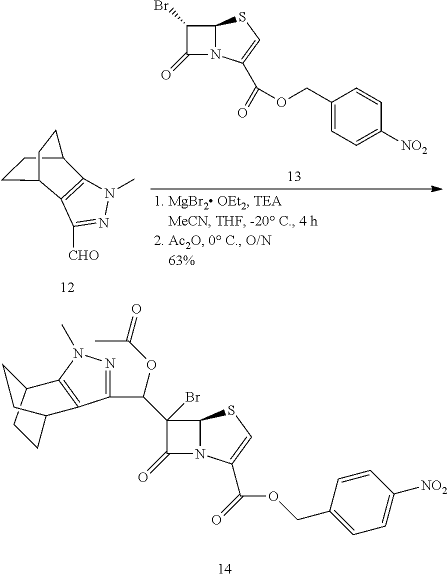

To a mixture of 1-methyl-4,5,6,7-tetrahydro-1H-4,7-ethanoindazole-3-carbaldehyde 12 (0.180 g, 0.950 mmol), 4-nitrobenzyl (5R,6S)-6-bromo-7-oxo-4-thia-1-azabicyclo[3.2.0]hept-2-ene-2-carboxylate 13 (0.400 g, 1.04 mmol) and magnesium bromide diethyl etherate (0.490 g, 1.90 mmol) in a solution of THF (15 mL) and acetonitrile (10 mL) was added Et₃N (0.264 mL, 1.90 mmol) at −20° C. under nitrogen. The resulting mixture was stirred at −20° C. for 4 h while the reaction flask was covered by aluminum foil to exclude light, and then acetic anhydride (0.449 mL, 4.75 mmol) was added. The resulting mixture was kept at 0° C. overnight and diluted with EtOAc, washed with 10% citric acid, saturated NaHCO₃ aqueous solution, brine, dried over Na₂SO₄ and concentrated to provide a residue, which was subjected to chromatography (silica gel, hexanes:EtOAc, 4:1 to 2:1) to give 14 (0.370 g, 63%) as a mixture of diastereoisomers.

Isomer 1:

¹H NMR (400 MHz, CDCl₃): δ 1.25 (2H, m), 1.72 (6H, m), 2.24 (3H, s), 3.20 (2H, d, J=1.6 Hz), 3.78 (3H, s), 5.25 (1H, d, J=13.6 Hz), 5.45 (1H, d, J=13.6 Hz), 5.99 (1H, s), 6.88 (1H, s), 7.45 (1H, s), 7.61 (2H, d, J=6.0 Hz), 8.24 (2H, d, J=6.0 Hz).

Isomer 2:

¹H NMR (400 MHz, CDCl₃): δ 1.24 (2H, m), 1.72 (6H, m), 2.05 (3H, s), 3.18 (1H, s), 3.32 (1H, s), 3.81 (3H, s), 5.26 (1H, d, J=13.2 Hz), 5.47 (1H, d, J=13.2 Hz), 6.26 (1H, s), 6.47 (1H, s), 7.47 (1H, s), 7.63 (2H, d, J=7.2 Hz), 8.24 (2H, d, J=7.2 Hz).

### Preparation of (5R,6Z)-6-[(1-methyl-4,5,6,7-tetrahydro-1H-4,7-ethanoindazol-3-yl)methylidene]-7-oxo-4-thia-1-azabicyclo[3.2.0]hept-2-ene-2-carboxylic acid (Compound D)

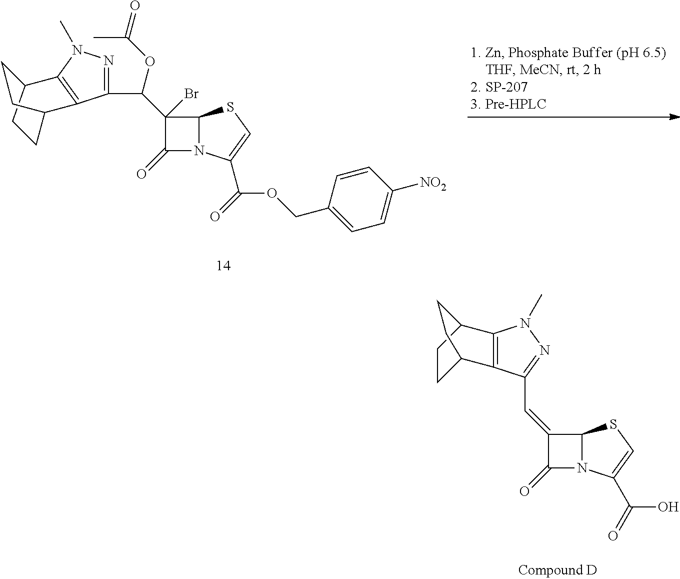

To a mixture of 4-nitrobenzyl (5R)-6-[(acetyloxy)(1-methyl-4,5,6,7-tetrahydro-1H-4,7-ethanoindazol-3-yl)methyl]-6-bromo-7-oxo-4-thia-1-azabicyclo[3.2.0]hept-2-ene-2-carboxylate 14 (mixture of 2 isomers, 0.18 g, 0.29 mmol) in a solution of MeCN (1.5 mL) and THF (3.0 mL) was added activated Zinc dust (2 g wet, activated by washing with 0.1 N HCl, water) followed by 0.5 M phosphate buffer (pH 6.5, 5.0 mL). The resulting mixture was vigorously stirred at room temperature for 2 h. The reaction flask was covered by aluminum foil to exclude light. After 2 h the reaction mixture was filtered through a pad of Celite. The filtrate was washed with EtOAc and the aqueous layer was separated. The aqueous layer was loaded over SP-207 resin column and eluted with water (750 mL) and with 20% acetonitrile in water. The yellow fractions were collected and concentrated under reduced pressure at 30° C. to remove organic solvent and freeze-dried to give product (40 mg, 40%) as sodium salt, which was further purified by preparative HPLC to give Compound B (13 mg) as a yellow solid.

¹H NMR (400 MHz, CDCl₃): δ 1.28 (4H, m), 1.77 (4H, m), 3.23 (2H, m), 3.88 (3H, s), 6.49 (1H, s), 7.09 (1H, s), 7.37 (1H, s).

HPLC purity: 84.69%.

MS (ES⁺) m/z: [M+H]⁺calcd for C₁₇H₁₇N₃O₃S: 344.11. Found: 344.20.

### Example 5

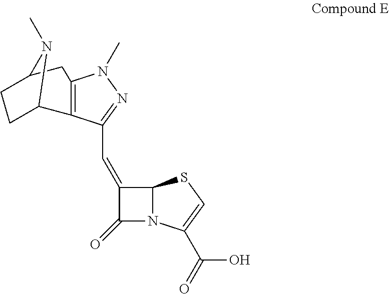

### Preparation of ethyl 1,9-dimethyl-1,4,5,6,7,8-hexahydro-4,7-epiminocyclohepta[c]pyrazole-3-carboxylate (3) and ethyl 2,9-dimethyl-2,4,5,6,7,8-hexahydro-4,7-epiminocyclohepta[c]pyrazole-3-carboxylate (4)

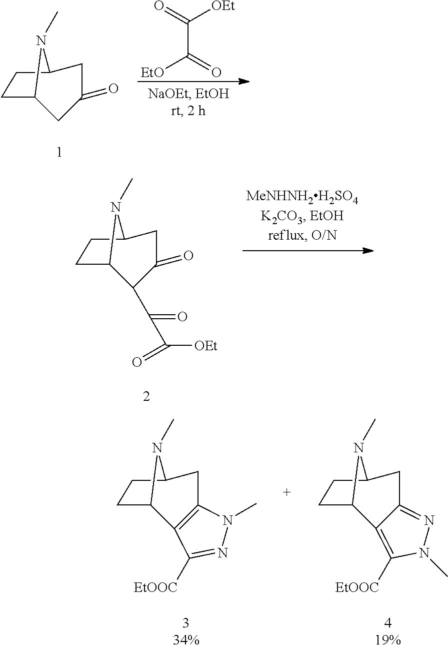

To a mixture of 8-methyl-8-azabicyclo[3.2.1]octan-3-one 1 (3.00 g, 21.6 mmol) and NaOEt (1.80 g, 25.6 mmol) in EtOH (35 mL) was added diethyl oxalate (2.92 mL, 21.6 mmol) slowly. The mixture was stirred at room temperature for 2 h. To this mixture were added methyl hydrazine sulfate (6.23 g, 43.2 mmol) and K₂CO₃ (5.96 g, 43.2 mmol). The resulting mixture was refluxed overnight and concentrated to provide a white solid, which was treated with water and EtOAc. The organic layer was separated and concentrated to give a residue, which was subjected to chromatography (silica gel, EtOAc:MeOH:NH₄OH (30%), 100:10:1) to provide 3 (1.84 g, 34%) as a yellow oil and 4 (1.00 g, 19%) as a yellow oil.

Compound 3:

¹H NMR (400 MHz, CDCl₃): δ 1.40 (3H, t, J=6.8 Hz), 1.43 (1H, m), 1.83 (1H, t, J=9.6 Hz), 2.23 (3H, m), 2.30 (3H, s), 2.96 (1H, dd, J=4.8 Hz, 16 Hz), 3.55 (1H, m), 3.79 (3H, s), 4.40 (3H, m).

Compound 4:

¹H NMR (400 MHz, CDCl₃): δ 1.39 (3H, m), 1.50 (1H, m), 1.76 (1H, m), 2.30 (3H, m), 2.34 (3H, s), 3.03 (1H, m), 3.54 (1H, m), 4.10 (3H, s), 4.35 (3H, m).

### Preparation of (1,9-dimethyl-1,4,5,6,7,8-hexahydro-4,7-epiminocyclohepta[c]pyrazol-3-yl)methanol (5)

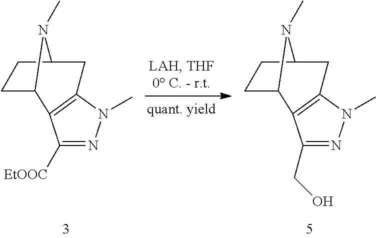

To a mixture of ethyl 1,9-dimethyl-1,4,5,6,7,8-hexahydro-4,7-epiminocyclohepta[c]pyrazole-3-carboxylate 3 (1.30 g, 5.20 mmol) in THF (40 mL) was added lithium aluminum hydride (1M in THF, 520 mL, 5.20 mmol) at 0° C. The reaction mixture was stirred at 0° C. for 1 h, quenched with NaOH aqueous solution, extracted with EtOAc and concentrated to provide 5 (1.10 g, quant. yield) as a yellow solid. The product was used in the next step without further purification.

¹H NMR (400 MHz, CDCl₃): δ 1.48 (1H, m), 1.79 (1H, m), 2.20 (3H, m), 2.29 (3H, s), 2.97 (1H, dd, J=4.0 Hz, 15.2 Hz), 3.52 (1H, m), 3.68 (3H, s), 3.98 (1H, m), 4.61 (2H, s).

### Preparation of 1,9-dimethyl-1,4,5,6,7,8-hexahydro-4,7-epiminocyclohepta[c]pyrazole-3-carbaldehyde (6)

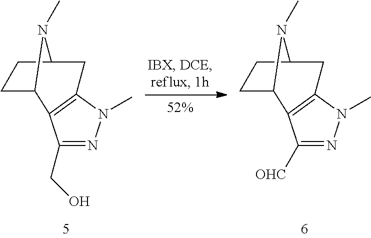

A mixture of (1,9-dimethyl-1,4,5,6,7,8-hexahydro-4,7-epiminocyclohepta[c]pyrazol-3-yl)methanol 5 (1.00 g, 5.20 mmol) and 1-hydroxy-1,2-benziodoxol-3H-one-1-oxide (2.18 g, 7.80 mmol) in dichloroethane (50 mL) was refluxed for 1 h, filtered through a pad of celite, washed with dichloromethane and concentrated to provide a residue, which was subjected to chromatography (silica gel, dichloromethane:MeOH, 10:1) to give 6 (0.77 g, 52%) as a yellow solid.

¹H NMR (400 MHz, CDCl₃): δ 1.45 (1H, m), 1.79 (1H, t, J=8.8 Hz), 2.24 (3H, m), 2.31 (3H, s), 2.98 (1H, dd, J=4.0 Hz, 16.0 Hz), 3.55 (1H, m), 3.81 (3H, s), 4.41 (1H, m), 9.94 (1H, s).

### Preparation of 4-nitrobenzyl (5R)-6-[(acetyloxy)(1,9-d ethyl-1,4,5,6,7,8-hexahydro-4,7-epiminocyclohepta[c]pyrazol-3-yl)methyl]-6-bromo-7-oxo-4-thia-1-azabicyclo[3.2.0]hept-2-ene-2-carboxylate (8)

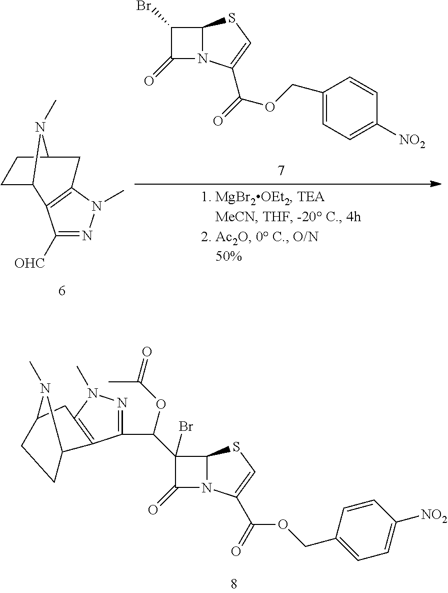

To a mixture of 1,9-dimethyl-1,4,5,6,7,8-hexahydro-4,7-epiminocyclohepta[c]pyrazole-3-carbaldehyde 6 (0.195 g, 0.950 mmol), 4-nitrobenzyl (5R,6S)-6-bromo-7-oxo-4-thia-1-azabicyclo[3.2.0]hept-2-ene-2-carboxylate 7 (0.400 g, 1.04 mmol) and magnesium bromide diethyl etherate (0.490 g, 1.90 mmol) in a solution of THF (15 mL) and acetonitrile (10 mL) was added Et₃N (0.264 mL, 1.90 mmol) at −20° C. under nitrogen. The resulting mixture was stirred at −20° C. for 4 h while the reaction flask was covered by aluminum foil to exclude light, and then acetic anhydride (0.449 mL, 4.75 mmol) was added. The resulting mixture was kept at 0° C. overnight and diluted with EtOAc, washed with 10% citric acid, saturated NaHCO₃ aqueous solution, brine, dried over Na₂SO₄ and concentrated to provide a residue, which was subjected to chromatography (silica gel, dichloromethane:MeOH, 100:5) to give 8 (0.300 g, 50%) as a mixture of diastereoisomers.

(ES⁺) m/z: [M+H]⁺calcd for C₂₆H₂₇BrN₅O₇S: 634.08, 632.08. Found: 634.15, 632.14.

### Preparation of (5R,6Z)-6-[(1,9-dimethyl-1,4,5,6,7,8-hexahydro-4,7-epiminocyclohepta[c]pyrazol-3-yl)methylidene]-7-oxo-4-thia-1-azabicyclo[3.2.0]hept-2-ene-2-carboxylic acid (Compound E)

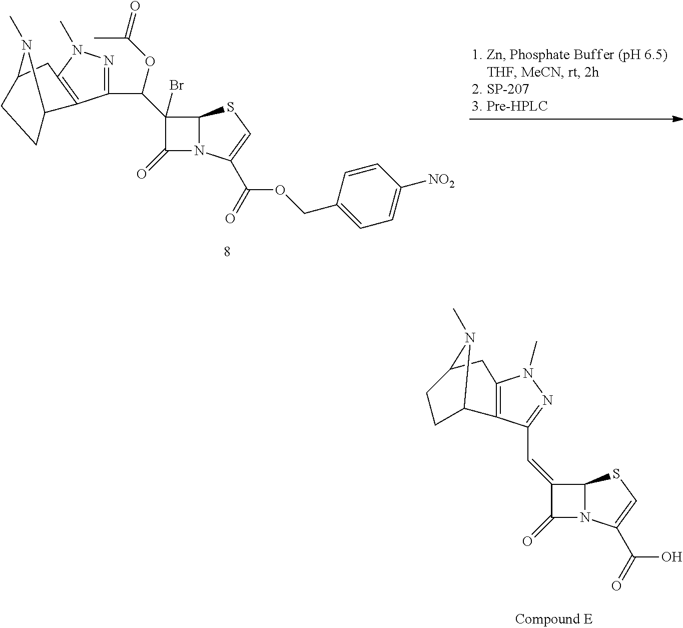

To a mixture of 4-nitrobenzyl (5R)-6-[(acetyloxy)(1,9-dimethyl-1,4,5,6,7,8-hexahydro-4,7-epiminocyclohepta[c]pyrazol-3-yl)methyl]-6-bromo-7-oxo-4-thia-1-azabicyclo[3.2.0]hept-2-ene-2-carboxylate 8 (0.16 g, 0.25 mmol) in a solution of MeCN (1.5 mL) and THF (3.0 mL) was added activated Zinc dust (3 g wet, activated by washing with 0.1 N HCl, water) followed by 0.5 M phosphate buffer (pH 6.5, 5.0 mL). The resulting mixture was vigorously stirred at room temperature for 2 h. The reaction flask was covered by aluminum foil to exclude light. After 2 h the reaction mixture was filtered through a pad of Celite. The filtrate was washed with EtOAc and the aqueous layer was separated. The aqueous layer was loaded over SP-207 resin column and eluted with water (750 mL) and with 20% acetonitrile in water. The yellow fractions were collected and concentrated under reduced pressure at 30° C. to remove organic solvent and freeze-dried to give product (20 mg) as sodium salt, which was further purified by preparative HPLC to give Compound E (6.0 mg) as a yellow solid.

¹H NMR (400 MHz, DMSO-d₆): δ 1.37 (1H, m), 1.58 (1H, m), 2.08 (3H, m), 2.12 (3H, m), 2.26 (1H, m), 2.87 (1H, m), 3.69 (3H, s), 4.08 (1H, m), 6.37 (1H, m), 7.04 (1H, m), 7.21 (1H, m).

HPLC purity: 92.02% (48.92%+44.01%, 2 isomers).

MS (ES⁺) m/z: [M+H]⁺ calcd for C₁₇H₁₉N₄O₃S: 359.12. Found: 359.17.

### Example 6

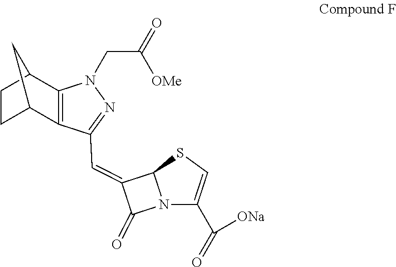

### Preparation of ethyl 4,5,6,7-tetrahydro-1H-4,7-methanoindazole-3-carboxylate (2)

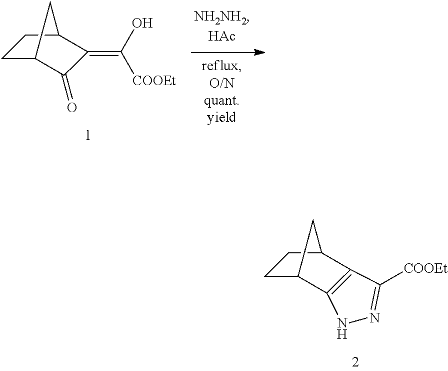

A mixture of ethyl 2-hydroxy(3-oxobicyclo[2.2.1]hept-2-ylidene)ethanoate 1 (8.10 g, 38.5 mmol) and hydrazine (2.26 mL, 46.2 mmol) in acetic acid (40 mL) was refluxed overnight and concentrated to provide a residue, which was treated with water and dichloromethane. The organic layer was washed with saturated sodium bicarbonate saturated solution, separated and concentrated to provide 2 (8.30 g, quant. yield) as a light brown oil.

¹H NMR (400 MHz, CDCl₃): δ 1.52 (2H, m), 1.36 (3H, t, J=7.6 Hz), 1.72 (1H, d, J=9.2 Hz), 1.96 (3H, m), 3.42 (1H, s), 3.57 (1H, s), 4.35 (2H, m).

### Preparation of 4,5,6,7-tetrahydro-1H-4,7-methanoindazol-3-ylmethanol (3)

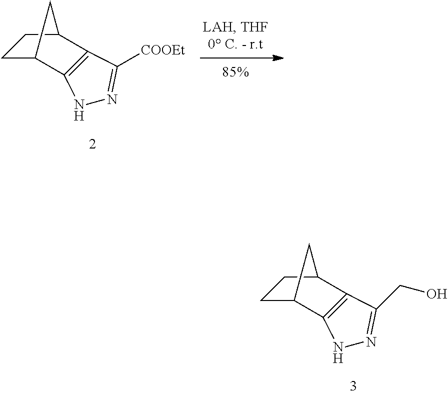

To a mixture of ethyl 4,5,6,7-tetrahydro-1H-4,7-methanoindazole-3-carboxylate 2 (0.488 g, 2.37 mmol) in THF (10 mL) was added lithium aluminum hydride (1M in THF, 2.37 mL, 2.37 mmol) at 0° C. The reaction mixture was stirred at 0° C. for 1 h, quenched with NaOH aqueous solution, extracted with EtOAc and concentrated to provide 3 (0.33 g, 85%) as a white solid. The product was used in the next step without further purification.

¹H NMR (400 MHz, CDCl₃): δ 1.24 (2H, m), 1.67 (1H, m), 1.91 (3H, m), 3.33 (1H, s), 3.37 (1H, s), 4.70 (2H, m).

### Preparation of methyl[3-(hydroxymethyl)-4,5,6,7-tetrahydro-4H-4,7-methanoindazol-1-yl]acetate (5)

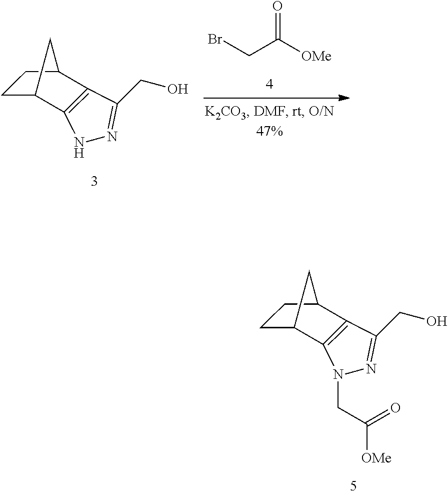

A mixture of 4,5,6,7-tetrahydro-1H-4,7-methanoindazol-3-ylmethanol 3 (2.11 g, 12.8 mmol), methyl bromoacetate 4 (1.46 mL, 15.4 mmol) and potassium carbonate (4.44 g, 32.1 mmol) in DMF (20 mL) was stirred at room temperature overnight. The reaction mixture was quenched with water, extracted with EtOAc and concentrated to give a residue, which was subjected to chromatography (silica gel, EtOAc: MeOH, 100:6) to provide 5 (1.45 g, 47%) as a yellow oil.

¹H NMR (400 MHz, CDCl₃): δ 1.18 (1H, m), 1.24 (1H, m), 1.62 (1H, d, J=8.8 Hz), 1.88 (2H, m), 2.00 (1H, d, J=8.8 Hz), 3.18 (1H, s), 3.39 (1H, s), 3.77 (3H, s), 4.63 (2H, d, J=1.6 Hz), 4.81 (2H, d, J=3.2 Hz).

### Preparation of methyl (3-formyl-4,5,6,7-tetrahydro-4H-4,7-methanoindazol-1-yl)acetate (6)

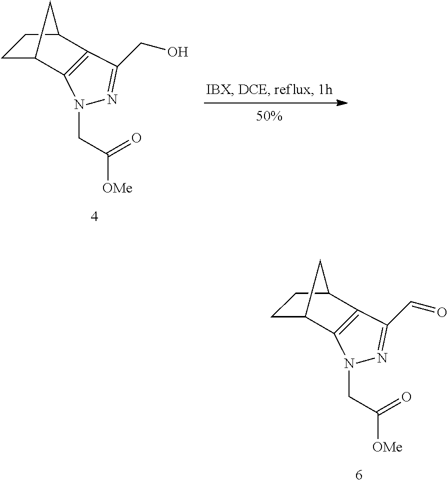

A mixture of methyl[3-(hydroxymethyl)-4,5,6,7-tetrahydro-1H-4,7-methanoindazol-1-yl]acetate 4 (0.310 g, 1.89 mmol) and 1-hydroxy-1,2-benziodoxol-3H-one-1-oxide (0.790 g, 2.83 mmol) in dichloroethane (20 mL) was refluxed for 1 h, filtered through a pad of celite, washed with dichloromethane and concentrated to provide a residue, which was subjected to chromatography (silica gel, hexanes:EtOAc, 2:1) to give 6 (0.22 g, 50%) as a white solid.

¹H NMR (400 MHz, CDCl₃): δ 1.26 (2H, m), 1.68 (1H, d, J=8.8 Hz), 1.94 (2H, m), 2.05 (1H, m), 3.37 (1H, s), 3.68 (1H, s), 3.81 (3H, s), 4.94 (2H, m).

### Preparation of 4-nitrobenzyl (5R)-6-{(acetyloxy)[1-(2-methoxy-2-oxoethyl)-4,5,6,7-tetrahydro-1H-4,7-methanoindazol-3-yl]methyl}-6-bromo-7-oxo-4-thia-1-azabicyclo[3.2.0]hept-2-ene-2-carboxylate (8)

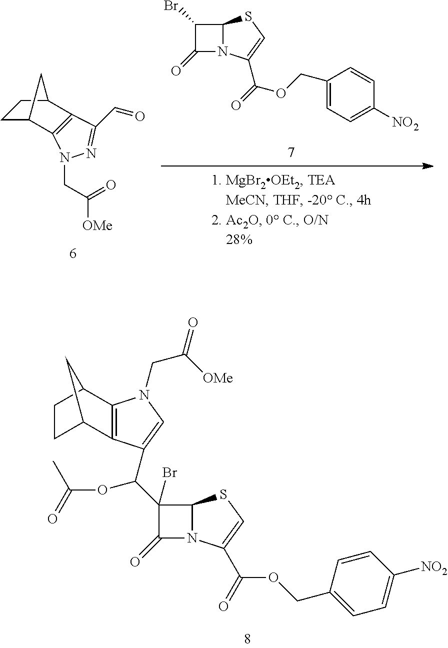

To a mixture of methyl (3-formyl-4,5,6,7-tetrahydro-1H-4,7-methanoindazol-1-yl)acetate 6 (0.202 g, 0.865 mmol), 4-nitrobenzyl (5R,6S)-6-bromo-7-oxo-4-thia-1-azabicyclo[3.2.0]hept-2-ene-2-carboxylate 7 (0.400 g, 1.04 mmol) and magnesium bromide diethyl etherate (0.668 g, 2.58 mmol) in a solution of TIE (12 mL) and acetonitrile (8 mL) was added Et₃N (0.241 mL, 1.73 mmol) at −20° C. under nitrogen. The resulting mixture was stirred at −20° C. for 4 h while the reaction flask was covered by aluminum foil to exclude light, and then acetic anhydride (0.408 mL, 4.32 mmol) was added. The resulting mixture was kept at 0° C. overnight and diluted with EtOAc, washed with 10% citric acid, sat. NaHCO₃ aqueous solution, brine, dried over Na₂SO₄ and concentrated to provide a residue, which was subjected to chromatography (silica gel, hexanes:EtOAc:THF, 400:100:50) to give 8 (0.16 g, 28%) as a mixture of diastereoisomers.

(ES⁺) m/z: [M+H]⁺ calcd for C₂₇H₂₆BrN₄O₉S: 663.06, 661.06. Found: 663.09, 661.09.

### Preparation of sodium (5R,6Z)-6-{[1-(2-methoxy-2-oxoethyl)-4,5,6,7-tetrahydro-1H-4,7-methanoindazol-3-yl]methylidene}-7-oxo-4-thia-1-azabicyclo[3.2.0]hept-2-ene-2-carboxylate (Compound F)

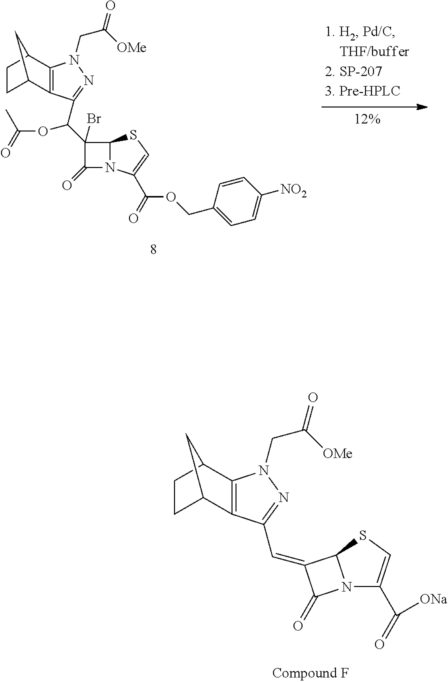

A mixture of 4-nitrobenzyl (5R)-6-{(acetyloxy)[1-(2-methoxy-2-oxoethyl)-4,5,6,7-tetrahydro-1H-4,7-methanoindazol-3-yl]methyl}-6-bromo-7-oxo-4-thia-1-azabicyclo[3.2.0]hept-2-ene-2-carboxylate 8 (0.16 g, 0.24 mmol) and Pd/C (5%, 0.15 g) in a solution of THF (15 mL) and sodium phosphate buffer solution (pH=6.5, 10 mL) was hydrogenated under 40 psi. After 2 h the reaction mixture was filtered through a pad of Celite. The filtrate was washed with EtOAc and the aqueous layer was separated. The aqueous layer was loaded over SP-207 resin column and eluted with water (750 mL) and with 20% acetonitrile in water. The yellow fractions were collected and concentrated under reduced pressure at 30° C. to remove organic solvent and freeze-dried to give product as sodium salt, which was further purified by preparative HPLC to give Compound F (12 mg, 12%) as a yellow solid. ¹H NMR (400 MHz, DMSO-d₆): δ 1.03 (1H, m), 1.14 (1H, m), 1.63 (1H, m), 1.86 (3H, m), 3.48 (2H, m), 3.70 (3H, s), 4.99 (1H, d, J=17.6 Hz), 5.10 (1H, d, J=17.6 Hz), 6.32 (0.75H, s), 6.45 (0.25H, s), 6.53 (0.75H, s), 6.55 (0.25H, s), 6.88 (0.25H, s), 6.89 (0.75H, s).

HPLC purity: 88.69%.

MS (ES⁺) m/z: [M+H]⁺ calcd for C₁₈H₁₇N₃O₅SNa: 410.08. Found: 410.12.

### Example 7

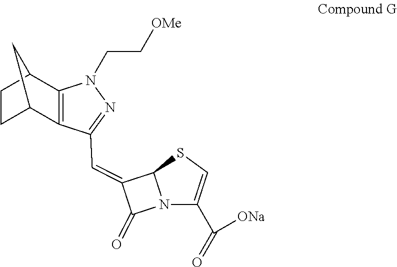

### Preparation of ethyl 1-(2-methoxyethyl)-4,5,6,7-tetrahydro-1H-4,7-methanoindazole-3-carboxylate (3) and ethyl 2-(2-methoxyethyl)-4,5,6,7-tetrahydro-2H-4,7-methanoindazole-3-carboxylate (4)

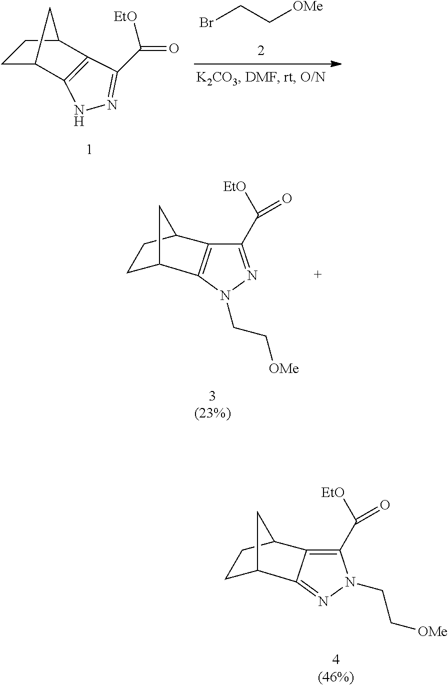

A mixture of ethyl 4,5,6,7-tetrahydro-1H-4,7-methanoindazole-3-carboxylate 1 (2.00 g, 9.70 mmol), 1-bromo-2-methoxyethane 2 (1.10 mL, 11.6 mmol) and potassium carbonate (2.68 g, 19.4 mmol) in DMF (6 mL) was stirred at room temperature overnight. The reaction mixture was quenched with water, extracted with EtOAc and concentrated to give a residue, which was subjected to chromatography (silica gel, EtOAc:Hexane, 1:4 to 1:2) to provide 3 (0.58 g, 23%) as a colorless oil and 4 (1.18 g, 46%) as a yellow oil.

Compound 3:

¹H NMR (400 MHz, CDCl₃): δ 1.15 (2H, d, J=6.8 Hz), 1.39 (3H, t, J=7.2 Hz), 1.65 (1H, m), 1.92 (2H, m), 2.04 (1H, m), 3.31 (3H, s), 3.42 (1H, s), 3.58 (1H, s), 3.72 (2H, m), 4.28 (2H, m), 4.38 (2H, m).

Compound 4:

¹H NMR (400 MHz, CDCl₃): δ 1.21 (2H, m), 1.36 (3H, t, J=7.2 Hz), 1.65 (1H, m), 1.94 (3H, m), 3.32 (3H, s), 3.39 (1H, s), 3.51 (1H, s), 3.71 (2H, t, J=5.6 Hz), 4.33 (2H, q, J=5.6 Hz), 4.55 (1H, m), 4.73 (1H, m).

### Preparation of [1-(2-methoxyethyl)-4,5,6,7-tetrahydro-1H-4,7-methanoindazol-3-yl]methanol (5)

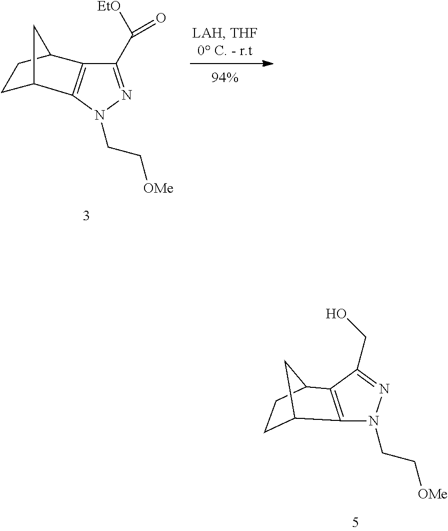

To a mixture of ethyl 1-(2-methoxyethyl)-4,5,6,7-tetrahydro-1H-4,7-methanoindazole-3-carboxylate 3 (0.580 g, 2.19 mmol) in THF (20 mL) was added lithium aluminum hydride (1M in THF, 2.19 mL, 2.19 mmol) at 0° C. The reaction mixture was stirred at room temperature for 1 h, quenched with NaOH aqueous solution, extracted with EtOAc and concentrated to provide 5 (0.46 g, 94%) as a colorless oil. The product was used in the next step without further purification.

¹H NMR (400 MHz, CDCl₃): δ 1.13 (2H, m), 1.63 (1H, m), 1.88 (2H, m), 1.97 (1H, 3.32 (3H, s), 3.36 (2H, m), 3.70 (2H, m), 4.17 (2H, m), 4.62 (2H, s).

### Preparation of 1-(2-methoxyethyl)-4,5,6,7-tetrahydro-4H-4,7-methanoindazole-3-carbaldehyde (6)

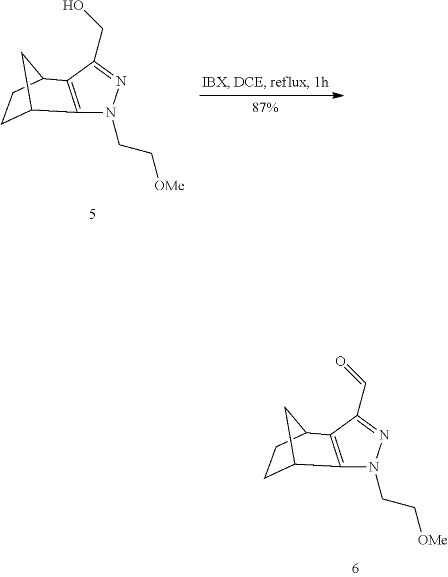

A mixture of [1-(2-methoxyethyl)-4,5,6,7-tetrahydro-1H-4,7-methanoindazol-3-yl]methanol 5 (0.460 g, 2.07 mmol) and 1-hydroxy-1,2-benziodoxol-3H-one-1-oxide (0.870 g, 3.10 mmol) in dichloroethane (25 mL) was refluxed for 1 h, filtered through a pad of celite, washed with dichloromethane and concentrated to provide a residue, which was subjected to chromatography (silica gel, hexanes:EtOAc, 4:1 to 2:1) to give 6 (0.40 g, 87%) as a yellow oil.

¹H NMR (400 MHz, CDCl₃): δ 1.11 (2H, m), 1.67 (1H, d, J=8.8 Hz), 1.93 (2H, d, J=8.8 Hz), 1.99 (1H, m), 3.32 (3H, s), 3.43 (1H, s), 3.65 (1H, s), 3.75 (2H, m), 4.30 (2H, t, J=5.2 Hz), 9.89 (1H, s).

### Preparation of 4-nitrobenzyl (5R)-6-{(acetyloxy)[1-(2-methoxyethyl)-4,5,6,7-tetrahydro-1H-4,7-methanoindazol-3-yl]methyl}-6-bromo-7-oxo-4-thia-1-azabicyclo[3.2.0]hept-2-ene-2-carboxylate (8)

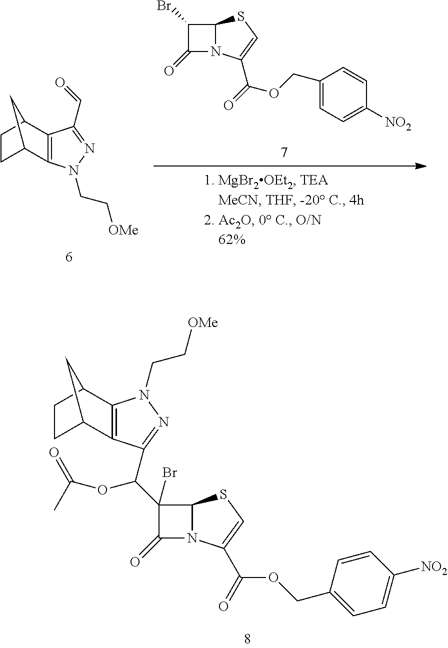

To a mixture of 1-(2-methoxyethyl)-4,5,6,7-tetrahydro-1H-4,7-methanoindazole-3-carbaldehyde 6 (0.10 g, 0.45 mmol), 4-nitrobenzyl (5R,6S)-6-bromo-7-oxo-4-thia-1-azabicyclo[3.2.0]hept-2-ene-2-carboxylate 7 (0.21 g, 0.54 mmol) and magnesium bromide diethyl etherate (0.332 g, 1.26 mmol) in a solution of THF (7.5 mL) and acetonitrile (5 mL) was added Et₃N (0.125 mL, 2.25 mmol) at −20° C. under nitrogen. The resulting mixture was stirred at −20° C. for 4 h while the reaction flask was covered by aluminum foil to exclude light, and then acetic anhydride (0.213 mL, 2.25 mmol) was added. The resulting mixture was kept at 0° C. overnight and diluted with EtOAc, washed with 10% citric acid, saturated NaHCO₃ aqueous solution, brine, dried over Na₂SO₄ and concentrated to provide a residue, which was subjected to chromatography (silica gel, hexanes:EtOAc:THF, 400:100:50) to give 8 (0.18 g, 62%) as a mixture of diastereoisomers.

(ES⁺) m/z: [M+H]⁺ calcd for C₂₇H₂₈BrN₄O₈S: 649.08, 647.08. Found: 649.06, 647.05.

### Preparation of sodium (5R,6Z)-6-{[1-(2-methoxyethyl)-4,5,6,7-tetrahydro-1H-4,7-methanoindazol-3-yl]methylidene}-7-oxo-4-thia-1-azabicyclo[3.2.0]hept-2-ene-2-carboxylate (Compound G)

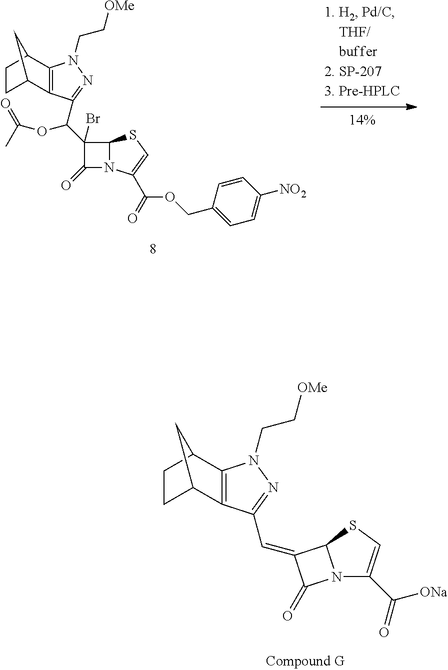

A mixture of 4-nitrobenzyl (5R)-6-{(acetyloxy)[1-(2-methoxyethyl)-4,5,6,7-tetrahydro-1H-4,7-methanoindazol-3-yl]methyl}-6-bromo-7-oxo-4-thia-1-azabicyclo[3.2.0]hept-2-ene-2-carboxylate 8 (0.36 g, 0.56 mmol) and Pd/C (5%, 0.20 g) in a solution of THF (30 mL) and sodium phosphate buffer solution (pH=6.5, 20 mL) was hydrogenated under 40 psi. After 2 h the reaction mixture was filtered through a pad of Celite. The filtrate was washed with EtOAc and the aqueous layer was separated. The aqueous layer was loaded over SP-207 resin column and eluted with water (750 mL) and with 20% acetonitrile in water. The yellow fractions were collected and concentrated under reduced pressure at 30° C. to remove organic solvent and freeze-dried to give product as sodium salt, which was further purified by preparative HPLC to give Compound G (32 mg, 14%) as a yellow solid.

¹H NMR (400 MHz, D₂O): δ 0.96 (2H, m), 1.65 (1H, m), 1.81 (3H, m), 3.17 (3H, s), 3.20 (0.5 H, s), 3.28 (0.5H, s), 3.37 (1H, s), 3.70 (2H, t, J=4.8 Hz), 4.15 (2H, m), 6.41 (0.5H, s), 6.47 (0.5H, s), 6.91 (2H, m).

HPLC purity: 99.28%.

MS (ES⁺) m/z: [M+H]⁺ calcd for C₁₈H₁₉N₃O₄SNa: 396.10. Found: 396.08.

What we claim:

1. A compound of formula (I) or formula (Ia) 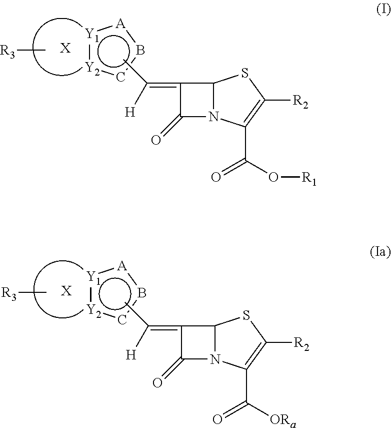
Wherein:
R₁ is the residue of a carboxy protecting group;
Rₐ is hydrogen or a pharmaceutically-acceptable salt forming agent or a pharmaceutically-acceptable ester residue readily hydrolyzable in vivo;
R₂ is selected from the group consisting of:
(a) Hydrogen,
(b) straight or branched chain alkyl,
(c) hydroxymethyl,
(d) alkoxymethyl,
(e) aminocarbonyloxymethyl,
(f) aryl,
(g) heteroaryl and
(h) heterocyclyl;
heteroaryl means a 5- or 6-membered unsaturated aromatic ring containing from 1 to 4 of any one or more of the hetero atoms selected from O, S and N; heterocyclyl means a 5-membered saturated ring containing one hetero atom;
X is a bridged bicyclic ring system having optionally one or two hetero atoms selected from O, S and N;
the ring X may be optionally substituted with R₃ wherein R₃ is selected from
(a) hydrogen,
(b) alkyl,
(c) hydroxy,
(d) alkoxy,
(e) hydroxymethyl,
(f) alkoxymethyl,
(g) halogen,
(h) cyano,
(i) carboxy,
(j) alkoxycarbonyl,
(k) amino,
(l) aminoalkyl,
(m) mono- or diallylamino,
(n) mono- or dialkylaminoalkyl,
(o) acylamino,
(p) sulfonylamino,
(q) substituted or unsubstituted amidino,
(r) substituted or unsubstituted urea,
(s) substituted or unsubstituted thiourea,
(t) substituted or unsubstituted carboxamido,
(u) substituted or unsubstituted thiocarboxamido,
(v) substituted or unsubstituted aryl,
(w) substituted or unsubstituted aralkyl,
(x) substituted or unsubstituted heteroaryl,
(y) substituted or unsubstituted heteroarylalkyl and
(z) substituted or unsubstituted heterocyclylalkyl;
the heteroaryl groups mentioned in items (x) and (y) means a 5- or 6-membered unsaturated aromatic ring containing from 1 to 4 of any one or more of the hetero atoms selected from O, S and N, wherein the said heteroaryl groups could be bonded via carbon, or a nitrogen-containing heteroaryl group could be bonded via nitrogen;
the bridged bicyclic ring systems containing a NH ring atom may be optionally substituted on the said nitrogen by a substituent selected from:
(a) alkyl,
(b) alkenyl,
(c) alkynyl,
(d) cycloalkyl,
(e) cycloalkylalkyl,
(f) cycloalkenyl,
(g) cycloalkenylalkyl,
(h) aryl,
(i) arylalkyl,
(j) heteroaryl,
(k) heteroarylalkyl,
(l) heterocyclyl,
(m) heterocyclylalkyl
(n) or a protecting group;
Y₁ and Y₂ may independently be C or N;
A, B or C form part of a heteroaryl ring where one of A, B or C is a carbon atom to which the remainder of the molecule is attached, and A, B and C are independently selected from CR₄, O, N, S or NR₅;
R₄ is hydrogen; and
R₅ is selected from the group consisting of:
(a) hydrogen,
(b) straight or branched lower alkyl,
(c) lower alkenyl,
(d) lower alkynyl,
(e) hydroxy alkyl,
(f) alkoxy alkyl,
(g) aminocarbonyloxy alkyl,
(h) cyano alkyl,
(i) aminoalkyl,
(j) mono- or dialkylaminoalkyl,
(k) alkoxycarbonylalkyl,
(l) carboxyalkyl,
(m) substituted or unsubstituted carboxamidoalkyl,
(n) cycloalkylalkyl,
(o) substituted or unsubstituted thiocarboxamidoalkyl,
(p) substituted or unsubstituted amidinoalkyl,
(q) substituted or unsubstituted guanidinoalkyl,
(r) substituted or unsubstituted aminocarbonylaminoalkyl,
(s) acylaminoalkyl,
(t) aralkyl,
(u) heteroarylalkyl and
(v) heterocyclylalkyl.

2. A compound according to claim 1, wherein R₁ can be removed without cleaving β-lactam ring, and is sufficiently stable under the reaction conditions to permit easy access to the compounds of formula (Ia) by de-esterification.

3. A compound according to claim 1, wherein R₁ is selected from the group consisting of 4-nitrobenzyl and 4-methoxybenzyl.

4. A pharmaceutical composition suitable for the treatment of bacterial infections in mammals comprising at least one compound recited in claim 1 and at least one pharmaceutically acceptable excipient.

5. A pharmaceutical composition according to claim 4, wherein said compound and β-lactam antibiotic are contained in the range of 1:20 to 20:1 weight ratios.

6. A method of treating bacterial infections, comprising administering to a subject in need of such treatment an effective amount of a β-lactam antibiotic and a compound according to claim 1.

7. A method as recited in claim 5, wherein the β-lactam antibiotic and the compound according to claim 1 are administered simultaneously.

8. A method as recited in claim 5, wherein the β-lactam antibiotic and the compound according to claim 1 are administered separately.

9. A method according to claim 5, wherein the β-lactam antibiotic is selected from the group consisting of amoxicillin, ampicillin, azlocillin, mezlocillin, apalcillin, hetacillin, bacampicillin, carbenicillin, sulbenicillin, ticarcillin, piperacillin, mecillinam, methicillin, ciclacillin, talampicillin, oxacillin, cloxacillin, dicloxacillin, cephalothin, cephaloridine, cefaclor, cefadroxil, cefamandole, cefazolin, cephalexin, cephradine, cephapirin, cefuroxime, cefoxitin, cephacetrile, cefotiam, cefotaxime, cefatriazine, cefsulodin, cefoperazone, ceftizoxime, cefmenoxime, cefmetazole, cephaloglycin, cefonicid, cefodizime, cefpirome, cefepime, ceftazidime, cefpiramide, ceftriaxone, cefbuperazone, cefprozil, cefixime, ceftobiprole, ceftaroline, cefalonium, cefminox, ceforanide, cefuzonam, cefoxitin, cefotetan, loracarbef, cefdinir, cefditoren, cefetamet, cefcapene, cefdaloxime, ceftibuten and cefroxamide.

10. A method for making a compound of formula (I) according to claim 1 comprising:
(a) Providing a compound of formula 1 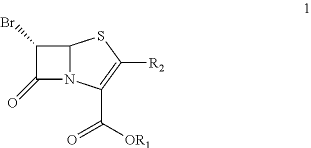
(b) Reacting the compound of formula I with R_{b}—CHO 2 to form a compound of formula 3; 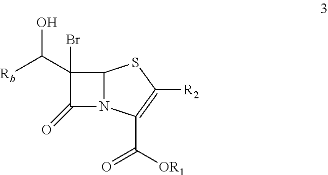
(c) Reacting the compound of formula 3 with acetic anhydride, trifluoromethane sulfonyl chloride or methane sulfonyl chloride to provide a compound of formula 4 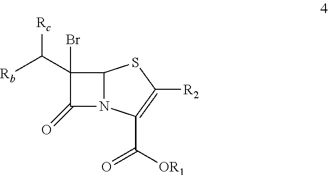
wherein R_{c} is selected from among OCOCH₃, OSO₂CF₃ and OSO₂CH₃;
(d) Treating the compound of formula 4 with activated zinc in presence of phosphate buffer to undergo reductive elimination with simultaneous deprotection of ester protecting group to provide a compound of formula 5, 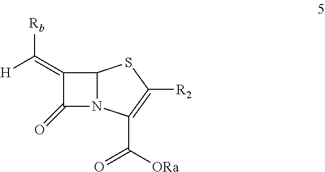
Wherein Rₐ is hydrogen, R_{b} is represented by the fragment (II) 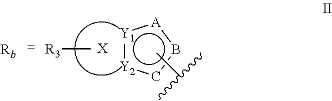
(e) and purifying the desired product.

11. A method of making a compound of formula (Ia) according to claim 1, comprising de-esterifying a compound of formula (I) to obtain a derivative of the formula (Ia) in which Rₐ is hydrogen.

12. A hydrate of a compound according to claim 1, wherein said hydrate contains variable amounts of water.

13. A hydrate according to claim 11, wherein said variable amounts of water result from lyophilization, crystallization or column purification.

14. A compound according to claim 1, wherein a regio-isomer is included.

15. A compound according to claim 1, wherein a stereoisomer is included.

16. A compound according to claim 1, wherein N-oxide is included.

17. A compound according to claim 1, wherein heterocyclyl means a 5-membered saturated ring containing oxygen.

18. A compound according to claim 1, wherein X is a bridged bicyclic ring system having optionally one or two hetero atoms selected from O and N.

19. A compound according to claim 1, wherein the heterocyclic rings under item (z) include:

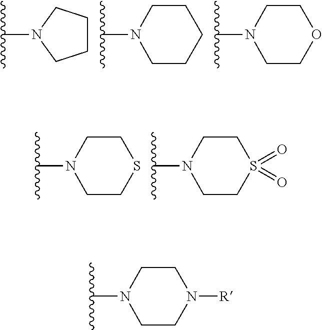

20. A compound according to claim 1, wherein the protecting group of item (n) for the optional substituent on the said nitrogen of the bridged bicyclic ring systems containing a NH ring atom is tert-butylcarbonyloxy.

21. A method according to claim 10, wherein reacting the compound of formula 3 to provide a compound of formula 4 comprises reacting the compound of formula 3 with acetic anhydride.
